(19) United States
(12) Patent Application Publication
(10) Pub. No.: US 2023/0044449 A1
(43) Pub. Date: Feb. 9, 2023

(54) NICLOSAMIDE FORMULATIONS AND METHODS OF USE AS CONTRACEPTIVE

(71) Applicant: YourChoice Therapeutics, Inc., Berkeley, CA (US)

(72) Inventors: Nadja MANNOWETZ, Berkeley, CA (US); Akash BAKSHI, Berkeley, CA (US); Andrew BARTYNSKI, Berkeley, CA (US)

(21) Appl. No.: 17/877,522

(22) Filed: Jul. 29, 2022

Related U.S. Application Data
(63) Continuation of application No. PCT/US2021/018880, filed on Feb. 19, 2021.
(60) Provisional application No. 62/980,104, filed on Feb. 21, 2020.

(51) Int. Cl. A61K 31/167; A61K 47/34; A61K 47/02; A61P 15/18

(52) U.S. Cl. CPC A61K 31/167; A61K 47/34; A61K 47/02; A61P 15/18

(57) ABSTRACT

Disclosed are formulations comprising niclosamide. The formulations disclosed may be used as a unisex and hormone-free on-demand contraceptive.

### RELATED APPLICATION

This application is a continuation of International Application No. PCT/US2021/018880, filed Feb. 19, 2021 which claims priority to and the benefit of U.S. Provisional Patent Application No. 62/980,104, filed on Feb. 21, 2020, the entire contents of which are incorporated herein by reference.

### FIELD OF THE DISCLOSURE

The present invention is directed to formulations of niclosamide for use in gels and creams and methods of using the same for contraception by decreasing sperm motility.

### BACKGROUND

Present contraceptive methods depend on hormonal control mechanisms or on physically disrupting contact between sperm cells and an egg using a barrier method. Women are more often the subject of these methods and, consequently, experience the negative side effects and secondary medical complications of systemic medication or implantation in the case of intrauterine devices. There is a long-felt and unmet need for non-hormonal and unisex, contraceptive compositions and methods that are effective, safe and easily applicable.

### SUMMARY

The disclosure provides a solution to this long-felt need as a formulation containing niclosamide that may be administered by either a men or women. Compositions and formulations of the disclosure may be easily applied by the subject and require no medical training to ensure proper use or effectiveness. Compositions and formulations of the disclosure are applied locally rather than systemically, eliminating the risk of drug interaction or long-term consequences of hormone dysregulation. Compositions and formulations of the disclosure may be effective “on demand,” meaning that the compositions and formulations of the disclosure need only be applied within minutes of sexual contact to be effective. This is in sharp contrast to a daily systemic medication or implant that remains in the body for months or years at a time.

The disclosure provides a formulation comprising niclosamide and a pharmaceutically-acceptable carrier.

The disclosure provides a formulation comprising niclosamide and one or more excipients. In some embodiments, the one or more excipients comprise one or more of a humectant, a lubricant, a solvent, an osmolality modulator, a pH modulator, a viscosity modulator, a preservative, or combinations thereof.

In some embodiments of the aqueous-based formulations of the disclosure, the formulation has a viscosity of between about 180 cPs and about 58,000 cPs. In some embodiments of the formulations of the disclosure, the formulation has a viscosity of between about 180 cPs and about 16,000 cPs. In some embodiments of the formulations of the disclosure, the formulation has a viscosity of between about 700 cPs and about 58,000 cPs. In some embodiments of the formulations of the disclosure, the viscosity modulator is hydroxyethyl cellulose (HEC), or carbopol, or polycarbophil, or pemulen. In some embodiments of the formulations of the disclosure, the formulation has a viscosity of between about 10,000 cPs and about 18,000 cPs. In some embodiments of the formulations of the disclosure, the viscosity modulator is carbopol or polycarbophil. In some embodiments of the formulations of the disclosure, the formulation has a viscosity of 11,000 cPs. In some embodiments of the formulations of the disclosure, the viscosity modulator is carbopol.

In some embodiments of the formulations of the disclosure, the humectant, the lubricant or the solvent comprise a synthetic polymer or water. In some embodiments of the formulations of the disclosure, the humectant, the lubricant or the solvent comprise a synthetic polymer. In some embodiments, the humectant, the lubricant or the solvent comprise at least two monomers of polyethylene glycol (PEG). In some embodiments of the formulations of the disclosure, the humectant, the lubricant or the solvent comprises at least two monomers of polyethylene glycol (PEG), optionally PEG-400, PEG-1000, or PEG-2000. In some embodiments, the humectant, the lubricant or the solvent comprise PEG-400. In some embodiments of the formulations of the disclosure, the formulation comprises an amount of PEG-400 between 0.1% and 75% of the total weight of the formulation, inclusive of the endpoints. In some embodiments of the formulations of the disclosure, the amount of PEG-400 is between 35% and 75% of the total weight of the formulation, inclusive of the endpoints. In some embodiments of the formulations of the disclosure, the amount of PEG-400 is between 35% and 65% of the total weight of the formulation, inclusive of the endpoints. In some embodiments of the formulations of the disclosure, the amount of PEG-400 is about 65% of the total weight of the formulation, inclusive of the endpoints. In some embodiments of the formulations of the disclosure, the amount of PEG-400 is 65% of the total weight of the formulation, inclusive of the endpoints.

In some embodiments, the formulation comprises an amount of the humectant, the lubricant or the solvent of between 0.1% and 65% of the total weight of the formulation, inclusive of the endpoints. In some embodiments, the solvent comprises water and the formulation comprise an amount of the water of between 10% and 90% of the total weight of the formulation, inclusive of the endpoints.

In some embodiments of the formulations of the disclosure, the formulations comprise a semi-solid form. In some embodiments, the semi-solid form comprises one or more of a gel, a hydrogel, a dehydrated gel, a hydrated gel, a paste, an ointment, a cream, and a lotion. In some embodiments, formulations of the disclosure comprise a gel or a cream.

In some embodiments of the formulations of the disclosure, the amount of niclosamide is between 0.1% and 10% of total weight of the formulation, inclusive of the endpoints. In some embodiments, the amount of niclosamide is between 0.1% and 5% of total weight of the formulation, inclusive of the endpoints. In some embodiments, the amount of niclosamide is between 1% and 5% of total weight of the formulation, inclusive of the endpoints. In some embodiments of the formulations of the disclosure, the amount of niclosamide is between 3% and 7% of total weight of the formulation, inclusive of the endpoints. In some embodiments of the formulations of the disclosure, the amount of niclosamide is between 3% and 6% of total weight of the formulation, inclusive of the endpoints. In some embodiments of the formulations of the disclosure, the amount of niclosamide is about 5% of total weight of the formulation. In some embodiments of the formulations of the disclosure, the amount of niclosamide is 5% of total weight of the formulation. In some embodiments, the amount of niclosamide is between 3% and 4% of total weight of the formulation, inclusive of the endpoints. In some embodiments, the amount of niclosamide is about 3.27% of total weight of the formulation. In some embodiments, the amount of niclosamide is 3.27% of total weight of the formulation.

In some embodiments of the formulations of the disclosure, the niclosamide has a concentration of between 4 mM and 500 mM, inclusive of the endpoints. In some embodiments, the niclosamide has a concentration of between 10 mM and 250 mM, inclusive of the endpoints. In some embodiments of the formulations of the disclosure, the niclosamide has a concentration of about 150 mM. In some embodiments of the formulations of the disclosure, the niclosamide has a concentration of 150 mM. In some embodiments, the niclosamide has a concentration of about 100 mM. In some embodiments, the niclosamide has a concentration of 100 mM.

In some embodiments of the formulations of the disclosure, the solvent comprises water, and wherein formulation comprises an amount of the water of between 10% and 90% of the total weight of the formulation, inclusive of the endpoints.

In some embodiments of the formulations of the disclosure, the osmolality modulator comprises sodium chloride. In some embodiments, the formulation comprises an amount of the osmolality modulator of less than 5% of the total weight of the formulation. In some embodiments, the formulation comprises an amount of the osmolality modulator of less than 1% of the total weight of the formulation.

In some embodiments of the formulations of the disclosure, the pH modulator comprises one or more of lactic acid, citric acid monohydrate, potassium sodium tartrate, potassium bitartrate and sodium hydroxide. In some embodiments, the formulation comprises an amount of the pH modulator of less than 10% of the total weight of the formulation and wherein the pH modulator comprise lactic acid, potassium sodium tartrate, citric acid monohydrate, potassium bitartrate, and sodium hydroxide. In some embodiments, the formulation comprises an amount of lactic acid of less than 5% of the total weight of the formulation and wherein the pH modulator comprises citric acid monohydrate, potassium sodium tartrate, potassium bitartrate, and sodium hydroxide. In some embodiments, the formulation comprises an amount of the pH modulator of less than 1% of the total weight of the formulation and wherein the pH modulator comprises one or more of potassium sodium tartrate, potassium bitartrate and sodium hydroxide. In some embodiments, the formulation comprises an amount of the pH modulator of less than 0.5% of the total weight of the formulation and wherein the pH modulator comprises one or more of potassium sodium tartrate, potassium bitartrate and sodium hydroxide.

In some embodiments of the formulations of the disclosure, the viscosity modulator is a viscosity enhancer. In some embodiments, the viscosity enhancer comprises one or more of hydroxyethyl cellulose, alginic acid, polycarbophil and carbopol. In some embodiments, the formulation comprises an amount of a viscosity enhancer of between 1% and 10% of the total weight of the formulation, inclusive of the endpoints. In some embodiments, the formulation comprises an amount of a viscosity enhancer of between 1% and 5% of the total weight of the formulation, inclusive of the endpoints. In some embodiments, the formulation comprises an amount of a viscosity enhancer of less than 5% of the total weight of the formulation. In some embodiments, the viscosity enhancer comprises one or more of hydroxyethyl cellulose, alginic acid, polycarbophil and carbopol. In some embodiments, the formulation comprises an amount of a viscosity enhancer of 3% of the total weight of the formulation. In some embodiments, the viscosity enhancer comprises hydroxyethyl cellulose. In some embodiments, the formulation comprises an amount of a viscosity enhancer less than 1% of the total weight of the formulation. In some embodiments, the formulation comprises an amount of a viscosity enhancer of about 0.5% of the total weight of the formulation. In some embodiments, the formulation comprises an amount of a viscosity enhancer of 0.5% of the total weight of the formulation. In some embodiments, the viscosity enhancer comprises carbopol. In some embodiments, the carbopol is carbopol-980.

In some embodiments, the preservative comprises one or more of benzyl alcohol, chlorhexidine gluconate and benzoic acid. In some embodiments, the preservative comprises benzyl alcohol or benzoic acid. In some embodiments, the preservative comprises benzyl alcohol. In some embodiments of the formulations of the disclosure, the formulation comprises an amount of the preservative of less than 1% of the total weight of the formulation. In some embodiments, the formulation comprises an amount of the preservative of about 0.2% of the total weight of the formulation. In some embodiments, the formulation comprises an amount of the preservative of 0.2% of the total weight of the formulation. In some embodiments, the formulation comprises an amount of the preservative of less than 0.2% of the total weight of the formulation. In some embodiments, the preservative comprises benzyl alcohol or benzoic acid. In some embodiments, the formulation comprises an amount of the preservative of less than 0.12% of the total weight of the formulation. In some embodiments, the preservative comprises chlorhexidine gluconate.

In some embodiments of the formulations of the disclosure, the formulation comprises, a) niclosamide at a concentration of 3% to 5% by weight of the formulation; b) a humectant, a lubricant or a solvent, wherein the humectant, the lubricant or the solvent comprises an amount of PEG-400 of 35% to 65% by weight of the formulation and an amount of water of 10% to 60% by weight of the formulation; c) an osmolality modulator, wherein the osmolality modulator comprises an amount of sodium chloride of 0.1% to 1% by weight of the formulation; d) at least one pH modulator, wherein the at least one pH modulator comprises an amount of lactic acid of 1% to 5% by weight of the formulation, an amount of citric acid monohydrate of 1% to 5% by weight of the formulation, an amount of potassium sodium tartrate of 0.1% to 0.5% by weight of the formulation, and an amount of sodium hydroxide of 0.1% to 0.5% by weight of the formulation; e) at least one viscosity modulator, wherein the at least one viscosity modulator comprises an amount of carbopol of 0.1% to 1% by weight of the formulation; and 0 at least one preservative, wherein the at least one preservative comprises an amount of benzyl alcohol of 0.1% to 0.5% by weight of the formulation.

In some embodiments of the formulations of the disclosure, the formulation comprises a) niclosamide at a concentration of 100 mM by weight of the formulation; b) a humectant, a lubricant or a solvent, wherein the humectant, the lubricant or the solvent comprises an amount of PEG-400 of between 0.1% and 65% by weight of the formulation and an amount of water of between 10% and 90% by weight of the formulation; c) an osmolality modulator, wherein the osmolality modulator comprise an amount of sodium chloride of less than 1% by weight of the formulation; d) at least one pH modulator, wherein the at least one pH modulator comprises san amount of lactic acid of less than 10% by weight of the formulation, an amount of citric acid monohydrate of less than 5% by weight of the formulation, an amount of potassium bitartrate of less than 0.5% by weight of the formulation, and an amount of sodium hydroxide of less than 1% by weight of the formulation; e) at least one viscosity modulator, wherein the at least one viscosity modulator comprises an amount of hydroxyethyl cellulose of 3% by weight of the formulation, an amount of alginic acid of less than 5% by weight of the formulation, an amount of polycarbophil of less than 5% by weight of the formulation and an amount of carbopol of less than 5% by weight of the formulation; and 0 at least one preservative, wherein the at least one preservative comprises an amount of benzyl alcohol of less than 1% by weight of the formulation, an amount of chlorhexidine gluconate of less than 0.12% by weight of the formulation, and an amount of benzoic acid of less than 0.2% by weight of the formulation.

The disclosure provides a method of contraception, comprising administering an effective amount of the formulation of the disclosure to the subject, wherein, upon contacting a sperm cell, the formulation inhibits or decreases one or more of sperm motility, sperm viability, and sperm metabolism, thereby decreasing the probability of conception.

In some embodiments of the methods of the disclosure, the formulation inhibits or decreases sperm motility by about 60%, 65%, 70%, 75%, 80%, 85%, 90%, 95%, 100%, or any percentage in between in comparison to a control value. In some embodiments, the control value is a predetermined value. In some embodiments, the control value is an average value of sperm motility measured from sperm cells obtained from one or more individual healthy donors. In some embodiments, sperm cells have not contacted the formulation. In some embodiments, the control value is determined in vitro.

In some embodiments of the methods of the disclosure, the formulation contacts a sperm cell for less than 2 minutes. In some embodiments, the formulation is administered to the subject less than 30 minutes prior to intercourse. In some embodiment, the formulation is administered to the subject between 15 and 20 minutes prior to intercourse.

In some embodiments of the methods of the disclosure, the formulation is administered locally. In some embodiments, the formulation is administered topically. In some embodiments, the subject is male. In some embodiments, the formulation is administered intravaginally. In some embodiments, the subject is female.

The disclosure provides a formulation of the disclosure for use in promoting contraception, comprising administering an effective amount of the formulation to the subject, wherein, upon contacting a sperm cell, the formulation inhibits or decreases one or more of sperm motility, sperm viability, and sperm metabolism, thereby decreasing the probability of conception.

In some embodiments of the formulations of the disclosure for use in promoting contraception, the formulation inhibits or decreases sperm motility by about 60%, 65%, 70%, 75%, 80%, 85%, 90%, 95%, 100%, or any percentage in between, in comparison to a control value. In some embodiments, the control value is a predetermined value. In some embodiments, the control value is an average value of sperm motility measured from sperm cells obtained from one or more healthy individual donors. In some embodiments, the sperm cells have not contacted the formulation. In some embodiments, the control value is determined in vitro. In some embodiments of the formulations of the disclosure for use in promoting contraception, the formulation inhibits or downregulates sperm viability by about 60%, 65%, 70%, 75%, 80%, 85%, 90%, 95%, 100%, or any percentage in between, in comparison to a control value. In some embodiments, the control value is a predetermined value. In some embodiments, the control value is an average value of sperm viability measured from healthy sperm cells obtained from one or more individual donors. In some embodiments, the healthy sperm cells have not contacted the formulation. In some embodiments, the control value is determined in vitro.

In some embodiments of the formulations of the disclosure for use in promoting contraception, the formulation inhibits or downregulates sperm metabolism as measured by the level of ATP in a semen sample by about 60%, 65%, 70%, 75%, 80%, 85%, 90%, 95%, 100%, or any percentage in between, in comparison, to a control value. In some embodiments, the control value is a predetermined value. In some embodiments, the control value is an average value of sperm metabolism as measured by the level of ATP in healthy semen samples obtained from one or more individual donors. In some embodiments, the healthy semen samples have not contacted the formulation. In some embodiments, the control value is determined in vitro.

In some embodiments, the therapeutically effective amount of the formulation of the disclosure is an amount that when contacted intravaginally to a female subject participating in sexual intercourse or coitus, before the sexual intercourse or coitus prevents or reduces the probability of conception, resulting from the sexual intercourse, activity or coitus, as compared to a female subject to whom the formulation of the disclosure has not been contacted intravaginally. In some embodiments, the therapeutically effective amount of the formulations of the disclosure, is an amount that reduces the the probability of conception in a female subject contacted with the formulation by at least 50%, at least 60%, at least 70%, at least 80%, at least 90%, a compared to a female subject to whom the formulation of the disclosure has not been contacted intravaginally. In some embodiments, the therapeutically effective amount of the formulations of the disclosure, is an amount that reduces the the probability of conception in a female subject contacted with the formulation by 100%, a compared to a female subject to whom the formulation of the disclosure has not been contacted intravaginally.

In some embodiments of the formulations of the disclosure for use in promoting contraception, the formulation contacts a sperm cell for less than 2 minutes. In some embodiments, the formulation is administered to the subject less than 30 minutes prior to intercourse. In some embodiment, the formulation is administered to the subject between 15 and 20 minutes prior to intercourse.

In some embodiments of the formulations of the disclosure for use in promoting contraception, the formulation is administered locally. In some embodiments, the formulation is administered topically. In some embodiments, the subject is male. In some embodiments, the formulation is administered intravaginally. In some embodiments, the subject is female.

The disclosure provides a method of decreasing the probability of conception in a female subject in need thereof, comprising contacting the female subject with an effective amount of the formulation of the disclosure, intravaginally prior to a sexual intercourse or coitus, wherein the probability of conceiving post the sexual intercourse or coitus of the female subject contacted with the formulation, is less than the probability of conceiving post the sexual intercourse or coitus of the female subject that has not been contacted with the formulation.

As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

### BRIEF DESCRIPTION OF THE DRAWINGS

FIG. 1 is a graph depicting the dose dependent effect of niclosamide dissolved in DMSO, on sperm motility. Fresh human semen samples were mixed with the different concentrations of niclosamide in vitro, followed by analysis of sperm motility within 2 minutes. The x-axis depicts the different concentrations of niclosamide mixed with the semen sample. The x-axis, from Left to Right: the gray bar depicts an untreated semen sample, the blue bar depicts a vehicle control, and the eight pink bars depict the increasing concentrations of niclosamide tested. There is 100% inhibition of sperm motility relative to the control treated sample at the highest doses of 10 mM and 50 mM niclosamide. The y-axis depicts the percentage (%) inhibition in sperm motility of each semen sample treated with a concentration of niclosamide o normalized to the sperm motility of a control sample. Change in sperm motility relative to control treated sample is shown as mean +/−standard error of the mean (S.E.M.) for the aggregate of individual experiments.

FIGS. 2A-2B are graphs comparing the ability of inhibiting sperm motility and pH buffering capacity of a niclosamide formulation of the disclosure with a commercially available contraceptive formulation, Phexxi. Fresh isolated human semen was mixed with either a niclosamide gel formulation (as depicted in Table 16, comprising 5% w/w niclosamide, 65% w/w PEG-400, 0.5% w/w carbopol 980 and 0.2% w/w benzyl alcohol), or Phexxi, as indicated in the graphs. FIG. 2A depicts the change in number of motile sperms (normalized to control) on the y-axis, and the corresponding human semen to formulation ratios on the x-axis, from left to right. FIG. 2B depicts the change in the pH of the semen and formulation mixture on the y-axis, and the corresponding human semen to formulation ratios on the x-axis, from left to right. Change in number of motile sperms and change in pH of the semen and formulation mixture are shown as mean +/−standard error of the mean (S.E.M.) for the aggregate of individual experiments.

FIGS. 3A-3C are haematoxylin and eosin stained images showing effect of niclosamide on vaginal tissue. Animals in groups G2 and G3 were treated with niclosamide at 2.5 and 100 mM/animal, respectively, and animals in group G1 were treated with vehicle control formulation, intra vaginally for 7 consecutive days (3F/group). On day 8, organs were collected (Cervical, central and caudal parts of the vagina) in 10% neutral buffer formalin solution, stained and analyzed for histpathology. FIG. 3A depicts Haematoxylin and Eosin Stained Images of vagina-cervical region (FIG. 3A), vagina-central region (FIG. 3B) and Vagina-caudal region (FIG. 3C) are shown.

### DETAILED DESCRIPTION

More adoptable contraceptives would be one that is: (1) easily acquired and administered, (2) inexpensive, (3) lacks systemic effects, (4) lacks the ability to cause irritation to the subject or his/her/their partner, (5) maintains normal flora and pH of the skin, vagina and/or rectum and, (6) provides contraceptive effects. Current contraceptive methods do not meet all of these requirements. Hormonal contraceptives, while convenient to use, have systemic effects and require regular visits to clinicians for the monitoring of possible severe side-effects (e.g. edema, weigh gain, abdominal bloating, nausea, depression, acne, hirsutism, vaginal bleeding, and adverse effects on plasma lipoprotein profiles). Barrier contraceptives for females such as the diagram and cervical caps require fitting by a clinician. Currently available and widely used spermicides have been shown to cause irritation in a large segment of the user population, due to the destruction of the normal vaginal flora, and increase susceptibility to sexually transmitted diseases and conditions caused by an increase of the vaginal pH.

Additional characteristics that have not yet before been sought after are contraceptive methods for male use. The formulations of the disclosure enable men to participate in contraception in a similarly safe, effective, and non-hormonal method. For use as a contraceptive the formulations of the disclosure may be applied topically to male subjects alone or in combination with a barrier method (e.g. a condom).

Non-hormonal formulations that can reduce or inhibit sperm functionality (motility and live sperm count) thereby effectively reducing or preventing sperm entry into the uterine tract and fusion with an ova, without negatively effecting the health and comfort of the subject, are highly desired.

The disclosure is directed to formulations, characterized by effective concentration ranges of niclosamide, and a pharmaceutically acceptable carrier, that effectively reduces or inhibits sperm motility, after contacting a sperm cell. The formulations of the disclosure may be in the form of a gel or cream formulation that is easily available and applicable by a subject.

Provided are formulations, comprising niclosamide, that can be in a gel or cream formulation, that completely inhibit sperm motility. Provided is a formulation comprising an amount of niclosamide and one or more of a humectant, a lubricant, a solvent, an osmolality modulator, a pH modulator, a viscosity modulator and a preservative. Provided is a method of contraception, comprising administering an effective amount of the formulation of the disclosure to the subject, wherein, upon contacting a sperm cell, the formulation inhibits or decreases one or more of sperm motility, sperm viability, and sperm metabolism, thereby decreasing the probability of conception. Provided is a formulation for use in a method of promoting contraception, wherein, the method comprises administering an effective amount of the formulation to the subject, wherein, upon contacting a sperm cell, the formulation inhibits or decreases one or more of sperm motility, sperm viability, and sperm metabolism, thereby decreasing the probability of conception. Provided is a use of the formulation of the disclosure, in the manufacture of a medicament for use in a method of promoting contraception in a subject.

In some embodiments of the formulations of the disclosure, the formulation has a viscosity of between about 180 cPs and about 58,000 cPs. In some embodiments of the formulations of the disclosure, the formulation has a viscosity of between about 180 cPs and about 16,000 cPs. In some embodiments of the formulations of the disclosure, the formulation has a viscosity of between about 700 cPs and about 58,000 cPs. In some embodiments of the formulations of the disclosure, the viscosity modulator is HEC, or carbopol, or polycarbophil, or pemulen. In some embodiments of the formulations of the disclosure, the formulation has a viscosity of between about 10,000 cPs and about 18,000 cPs. In some embodiments of the formulations of the disclosure, the viscosity modulator is carbopol or polycarbophil. In some embodiments of the formulations of the disclosure, the formulation has a viscosity of 7,00 cPs. In some embodiments of the formulations of the disclosure, the viscosity modulator is HEC. In some embodiments of the formulations of the disclosure, the formulation has a viscosity of 11,000 cPs. In some embodiments of the formulations of the disclosure, the viscosity modulator is carbopol. In some embodiments of the formulations of the disclosure, the formulation has a viscosity of 18,000 cPs. In some embodiments of the formulations of the disclosure, the viscosity modulator is polycarbophil. In some embodiments of the formulations of the disclosure, the formulation has a viscosity of 58,000 cPs. In some embodiments of the formulations of the disclosure, the viscosity modulator is pemulen.

### Niclosamide

Formulations of the disclosure comprise an amount of niclosamide.

Niclosamide has the chemical formula: C₁₃H₈C₁₂N₂O₄, molecular weight of 327.12 g/mol and the chemical structure:

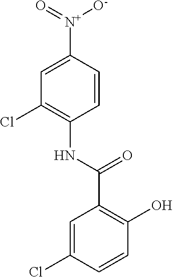 [SMILES: O=C(Nc1ccc([N+](=O)[O-])cc1Cl)c1cc(Cl)ccc1O]

The use of nicosamide had been approved by the United States Federal Drug Administration (FDA) under the trade name “NICLOCIDE” as an orally administrated chewable tablet having 500 mg of the niclosamide active ingredient for the treatment of tapeworm infections. NICLOCIDE has been discontinued.

Formulation of niclosamide as a semi-solid or liquid presents unique challenges as niclosamide is difficult to maintain in solution or in any form other than a dry solid. The formulations of the disclosure not only overcome this challenge, but also provide a new route of administration and new uses of the formulations as a contraceptive for use by either a man or a woman. Thus, the formulations of the disclosure provide a unisex contraceptive formulation. Route of administration may vary depending on the sex of the subject using the formulation. However, the formulations of the disclosure are safe and effective for local administration, either topically or internally, when applied to a surface of a body cavity.

### Niclosamide Formulations

Formulations of the disclosure, including a semi-solid form, a gel, a lotion or a cream, may comprise a concentration of the niclosamide of between 1 mM and 10 mM, 10 mM and 100 mM, 10 mM and 20 mM, 20 mM and 30 mM, 30 mM and 40 mM, 40 mM and 50 mM, 50 mM and 60 mM, 60 mM and 70 mM, 70 mM and 80 mM, 80 mM and 90 mM, 90 mM and 100 mM, inclusive of the endpoints. In some embodiments, formulations of the disclosure may comprise a concentration of niclosamide of 4 mM. In some embodiments, formulations of the disclosure may comprise a concentration of niclosamide of 100 mM. In some embodiments, formulations of the disclosure may comprise a concentration of niclosamide of between 4 mM and 100 mM, inclusive of the endpoints.

In some embodiments of the formulations of the disclosure, including a semi-solid form, a gel, a lotion or a cream, the formulation may comprise an amount of niclosamide, of between 0.1% and 10%, of the total weight of the formulation, inclusive of the endpoints. In some embodiments of the formulations of the disclosure, including a semi-solid form, a gel, a lotion or a cream, the formulation may comprise an amount of niclosamide, of between 0.1% and 10%, 0.1% and 9%, 0.1% and 8%, 0.1% and 7%, 0.1% and 6%, 0.1% and 5%, 0.1% and 4%, 0.1% and 3%, 0.1% and 2%, and 0.1% and 1%, of the total weight of the formulation, inclusive of the endpoints. In some embodiments of the formulations of the disclosure, including a semi-solid form, a gel, a lotion or a cream, the formulations may comprise an amount of niclosamide, of between 0.1% and 1%, 0.1% and 0.9%, 0.1% and 0.8%, 0.1% and 8%, 0.1% and 0.7%, 0.1% and 0.6%, 0.1% and 0.5%, 0.1% and 0.4%, 0.1% and 0.3%, 0.1% and 0.2%, of the total weight of the formulation, inclusive of the endpoints. In some embodiments of the formulations of the disclosure, including a semi-solid form, a gel, a lotion or a cream, the formulation may comprise an amount of niclosamide, between 1% and 10%, 1% and 9%, 1% and 8%, 1% and 7%, 1% and 6%, 1% and 5%, 1% and 4%, 1% and 3%, 1% and 2%, of the total weight of the formulation, inclusive of the endpoints. In some embodiments of the formulations of the disclosure, the formulation may comprise an amount of the niclosamide, between 1% and 5% of the total weight of the formulation, including the end points. In some embodiments of the formulations of the disclosure, the formulation may comprise an amount of niclosamide, of 0.13% of the total weight of the formulation. In some embodiments of the formulations of the disclosure, the formulation may comprise an amount of niclosamide, of 3.27% of the total weight of the formulation. In some embodiments of the formulations of the disclosure, the formulation may comprise an amount of niclosamide, of 10% of the total weight of the formulation.

In some embodiments of the formulations of the disclosure, the formulation may comprise an amount of niclosamide, of between 0.1% and 10% of total weight of the formulation, inclusive of the endpoints. In some embodiments of the formulations of the disclosure, the formulation may comprise an amount of niclosamide, of between 0.1% and 5% of total weight of the formulation, inclusive of the endpoints. In some embodiments of the formulations of the disclosure, the formulation may comprise an amount of niclosamide, of between 1% and 5% of total weight of the formulation, inclusive of the endpoints. In some embodiments of the formulations of the disclosure, the formulation may comprise an amount of niclosamide, of between 3% and 4% of total weight of the formulation, inclusive of the endpoints. In some embodiments of the formulations of the disclosure, the amount of niclosamide, is between 3% and 7% of total weight of the formulation, inclusive of the endpoints. In some embodiments of the formulations of the disclosure, the amount of niclosamide, is between 3% and 6% of total weight of the formulation, inclusive of the endpoints. In some embodiments of the formulations of the disclosure, the amount of niclosamide, is about 5% of total weight of the formulation. In some embodiments of the formulations of the disclosure, the amount of niclosamide, is 5% of total weight of the formulation. In some embodiments of the formulations of the disclosure, the amount of niclosamide is about 3% of total weight of the formulation. In some embodiments of the formulations of the disclosure, the amount of niclosamide is 3% of total weight of the formulation. In some embodiments of the formulations of the disclosure, the formulation may comprise an amount of niclosamide, of about 3.27% of total weight of the formulation. In some embodiments of the formulations of the disclosure, the formulation may comprise an amount of niclosamide, of 3.27% of total weight of the formulation. In some embodiments of the formulations of the disclosure, the amount of niclosamide is 1% to 2%, 2% to 3%, 3% to 4% or 4% to 5% of total weight of the formulation, inclusive of the endpoints.

In some embodiments of the formulations of the disclosure, the formulation may comprise niclosamide, at a concentration that is between 4 mM and 500 mM, inclusive of the endpoints. In some embodiments of the formulations of the disclosure, the formulation may comprise niclosamide, at a concentration that is between 10 mM and 250 mM, inclusive of the endpoints. In some embodiments of the formulations of the disclosure, the niclosamide has a concentration of about 150 mM. In some embodiments of the formulations of the disclosure, the niclosamide has a concentration of between 5 mM to 25 mM, 25 mM to 50 mM, 50 mM to 75 mM, 75 mM to 100 mM, 100 mM to 125 mM, 125 mM to 150 mM, 150 mM to 175 mM, 175 mM to 200 mM, 200 mM to 225 mM, 225 mM to 250 mM, 250 mM to 275 mM, 275 mM to 300 mM, 300 mM to 325 mM, 325 mM to 350 mM, 350 mM to 375 mM, 375 mM to 400 mM, 400 mM to 425 mM, 425 mM to 450 mM, 450 mM to 475 mM or 475 mM to 500 mM, inclusive of the endpoints. In some embodiments of the formulations of the disclosure, the niclosamide has a concentration of about 150 mM. In some embodiments of the formulations of the disclosure, the niclosamide has a concentration of 150 mM. In some embodiments of the formulations of the disclosure, the formulation may comprise niclosamide, at a concentration of about 100 mM. In some embodiments of the formulations of the disclosure, the formulation may comprise niclosamide, at a concentration of 100 mM. In some embodiments of the formulations of the disclosure, the niclosamide formulation may be a gel or a cream administered by a local route (e.g., topically or intra-cavitally to a component of the male or female reproductive system) and at a concentration of 150 nM.

In some embodiments of the formulations of the disclosure, including a semi-solid form, a gel, a lotion or a cream, the formulation may comprise an amount of niclosamide, at the concentration or percentage by weight values, as depicted in Tables 1 and 2.

In some embodiments of the formulations of the disclosure, including a semi-solid form, a gel, a lotion or a cream, the formulation one or more of a filler, an excipient, an anti-adherent, a humectant, a coloring agent, a glidant, a preservative, a sorbent, a bulking agent, a lubricating agent, an osmolality adjusting agent, an anti-oxidant, a vehicle, a binding agent, a disintegration agent, a buffering agent, a solvent, a viscosity agent, and a stability agent.

In some embodiments of the formulations of the disclosure, including a semi-solid form, a gel, a lotion or a cream, the formulation a humectant or a solvent including, but not limited to, saccharides and their derivatives (e.g., disaccharides: sucrose, lactose), polysaccharides and their derivatives (e.g., starches, cellulose or modified cellulose such as microcrystalline cellulose and cellulose ethers such as hydroxypropyl cellulose (HPC)), sugar alcohols (e.g., xylitol, sorbitol or maltitol), protein gelatin, and synthetic polymers (e.g., polyvinylpyrrolidone (PVP), polyethylene glycol (PEG)).

In some embodiments, the humectant, the lubricant or the solvent of the formulation of the disclosure comprise a synthetic polymer or water. In some embodiments, the humectant, the lubricant or the solvent comprise at least two monomers of polyethylene glycol (PEG). In some embodiments, the formulation may comprise a polyethylene glycol (PEG). In some embodiments of the formulations of the disclosure, the humectant, the lubricant or the solvent comprises at least two monomers of polyethylene glycol (PEG), optionally PEG-400, PEG-1000, or PEG-2000. In some embodiments, the humectant, the lubricant or the solvent comprise PEG-400. In some embodiments of the formulations of the disclosure, the formulation comprises an amount of PEG-400 between 0.1% and 75% of the total weight of the formulation, inclusive of the endpoints. In some embodiments of the formulations of the disclosure, the amount of PEG-400 is between 35% and 75% of the total weight of the formulation, inclusive of the endpoints. In some embodiments of the formulations of the disclosure, the amount of PEG-400 is between 35% and 65% of the total weight of the formulation, inclusive of the endpoints. In some embodiments of the formulations of the disclosure, the amount of PEG-400 is about 35% of the total weight of the formulation. In some embodiments of the formulations of the disclosure, the amount of PEG-400 is 35% of the total weight of the formulation. In some embodiments of the formulations of the disclosure, the amount of PEG-400 is about 65% of the total weight of the formulation. In some embodiments of the formulations of the disclosure, the amount of PEG-400 is 65% of the total weight of the formulation.

In some embodiments the formulation of the disclosure, the formulation comprises an amount of the humectant, the lubricant or the solvent of between 0.1% and 65% of the total weight of the formulation, inclusive of the endpoints. In some embodiments the formulation of the disclosure, the formulation comprises a solvent, wherein the solvent comprises water and wherein formulation comprises an amount of the water of between 10% and 90% of the total weight of the formulation, inclusive of the endpoints.

In some embodiments the formulation of the disclosure, the formulation comprises PEG-400 between 0.1% and 75%, 0.1% and 65%, 0.1% and 60%, 0.1% and 55%, 0.1% and 50%, 0.1% and 50%, 0.1% and 45%, 0.1% and 40%, 0.1% and 35%, 0.1% and 30%, 0.1% and 20%, 0.1% and 10% or 0.1% and 5%, inclusive of the endpoints. In some embodiments the formulation of the disclosure, the formulation comprises PEG-400 between 1% to 10%, 20% and 30%, 30% and 40%, 40% and 50%, 50% and 60%, 60% and 70%, 70% and 80% or 80% and 90%, inclusive of the endpoints. In some embodiments the formulation of the disclosure, the formulation comprises PEG-400 between 5% to 10%, 10% and 15%, 15% and 20%, 20% and 25%, 25% and 30%, 30% and 35%, 35% and 40%, 40% and 45%, 45% and 50%, 50% and 55%, 55% and 60%, 60% and 65%, 65% and 70% or 70% and 75%, inclusive of the endpoints.

In some embodiments the formulation of the disclosure, the formulation comprises PEG-400, of the amount or percentage by weight values, as depicted in Tables 1 and 2.

In some embodiments, the formulation comprises an amount of the humectant, the lubricant or the solvent of between 0.1% and 65% of the total weight of the formulation, inclusive of the endpoints. In some embodiments, the solvent comprises water and the formulation comprise an amount of the water of between 10% and 90% of the total weight of the formulation, inclusive of the endpoints. In some embodiments, the solvent comprises water and the formulation comprise an amount of the water of between 10% and 20%, 20% and 30%, 30% and 40%, 40% and 50%, 50% and 60%, 60% and 70%, 70% and 80% or 80% and 90% of the total weight of the formulation, inclusive of the endpoints. In some embodiments, the water is purified water.

In some embodiments the formulation of the disclosure, the formulation comprise an osmolality adjusting agent or osmolality modulator. In some embodiments, the formulations of the disclosure comprise an osmolality modulator selected from the group consisting of salt of aluminum, arginine, benzathine, calcium, chloroprocaine, choline, diethanolamine, ethanolamine, ethylenediamine, histidine, lithium, lysine, magnesium, meglumine, potassium, procaine, sodium, triethylamine, zinc or a combination thereof. In some embodiments, the osmolality modulator is a salt selected from the group consisting of acetate, glutamate, mucate, aspartate, glycolate, napsylate, benzenesulfonate, glycollylarsanilate, nitrate, benzoate, hexanoate, octanoate, chloroprocaine, besylate, hexylresorcinate, oleate, bicarbonate, hydrabamine, pamoate, bitartrate, hydroxynaphthoate, pantothenate, bromide, iodide, phosphate, camsylate, isethionate, polygalacturonate, carbonate, isethionate, propionate, chloride, lactate, salicylate, citrate, lactobionate, stearate, decanoate, malate, subacetate, edetate, maleate, succinate, estolate, mandelate, sulfate, esylate, mesylate, tartrate, fumarate, methylbromide, teoclate, gluceptate, methylnitrate, tosylate, gluconate, methylsulfate, triethiodide or a combination thereof.

In some embodiments of the formulations of the disclosure, the osmolality modulator is sodium chloride. In some embodiments, the formulation comprises an amount of the osmolality modulator of less than 5% of the total weight of the formulation. In some embodiments, the formulation comprises an amount of the osmolality modulator of less than 1% of the total weight of the formulation.

In some embodiments, the formulations of the disclosure, comprise sodium chloride, in an amount of 0.9%, 0.8%, 0.7%, 0.6%, 0.5%, 0.4%, 0.3%, 0.2% or 0.1%, of the total weight of the formulation.

In some embodiments, the formulations of the disclosure, comprise sodium chloride in an amount or percentage by weight, as depicted Tables 1 and 2.

In some embodiments, the formulations of the disclosure, comprise a pH modulator. In some embodiments, the formulations of the disclosure, comprise a pH modulator, wherein the pH modulator comprise one or more of lactic acid, citric acid monohydrate, potassium sodium tartrate, potassium bitartrate, and sodium hydroxide. In some embodiments, the formulations of the disclosure, comprise an amount of the pH modulator of less than 10% of the total weight of the formulation and wherein the pH modulator comprises lactic acid. In some embodiments, the formulations of the disclosure, comprise an amount of lactic acid of less than 5% of the total weight of the formulation and wherein the pH modulator comprises lactic acid or citric acid monohydrate. In some embodiments, the formulations of the disclosure, comprise an amount of the pH modulator of less than 1% of the total weight of the formulation and wherein the pH modulator comprises one or more of lactic acid, citric acid monohydrate, and sodium hydroxide. In some embodiments, the formulations of the disclosure, comprise an amount of the pH modulator of less than 0.5% of the total weight of the formulation and wherein the pH modulator comprises one or more of lactic acid, citric acid monohydrate, potassium bitartrate and sodium hydroxide.

In some embodiments of the formulations of the disclosure, the pH modulator comprises one or more of lactic acid, citric acid monohydrate, potassium sodium tartrate, potassium bitartrate and sodium hydroxide. In some embodiments, the formulation comprises an amount of the pH modulator of less than 10% of the total weight of the formulation and wherein the pH modulator comprises lactic acid, potassium sodium tartrate, citric acid monohydrate, potassium bitartrate and sodium hydroxide. In some embodiments, the formulation comprises an amount of pH modulator of less than 5% of the total weight of the formulation and wherein the pH modulator comprises citric acid monohydrate, potassium sodium tartrate, potassium bitartrate and sodium hydroxide. In some embodiments, the formulation comprises an amount of the pH modulator of less than 1% of the total weight of the formulation and wherein the pH modulator comprises one or more of potassium sodium tartrate, potassium bitartrate and sodium hydroxide. In some embodiments, the formulation comprises an amount of the pH modulator of less than 0.5% of the total weight of the formulation and wherein the pH modulator comprises one or more of potassium sodium tartrate, potassium bitartrate and sodium hydroxide.

In some embodiments, the formulations of the disclosure, comprise a pH modulating agent or buffer that is lactic acid. In some embodiments, the formulation of the disclosure comprise lactic acid in an amount of less than 10%, 9%, 8%, 7%, 6%, 5%, 4%, 3%, 2% or 1%, of the total weight of the formulation, including the end points. In some embodiments, the formulation of the disclosure , including a semi-solid form, a gel, a lotion or a cream, comprise lactic acid, in an amount of less than 10% of the total weight of the formulation.

In some embodiments, the formulations of the disclosure, comprise a pH modulating agent or buffer that is citric acid monohydrate. In some embodiments, the formulations of the disclosure, comprise citric acid monohydrate, in an amount of less than 5%, 4%, 3%, 2%, 1%, 0.9%, 0.8%, 0.7%, 0.6%, 0.5%, 0.4%, 0.3%, 0.2% or 0.1%, of the total weight of the formulation, including the end points. In some embodiments, the formulations of the disclosure, comprise citric acid monohydrate, in an amount of less than 5% of the total weight of the formulation.

In some embodiments, the formulations of the disclosure comprise a pH modulating agent or buffer that is potassium sodium tartrate. In some embodiments, the formulation of the disclosure comprise potassium sodium tartrate, in an amount of less than 0.5%, 0.4%, 0.3%, 0.2%, 0.1%, 0.09%, 0.08%, 0.07%, 0.06%, 0.05%, 0.04%, 0.03%, 0.02% or 0.01%, of the total weight of the formulation, including the end points. In some embodiments, the formulations of the disclosure, comprise potassium sodium tartrate, in an amount of less than 0.5% of the total weight of the formulation.

In some embodiments, the formulations of the disclosure comprise a pH modulating agent or buffer that is potassium bitartrate. In some embodiments, the formulation of the disclosure comprise potassium bitartrate, in an amount of less than 0.5%, 0.4%, 0.3%, 0.2%, 0.1%, 0.09%, 0.08%, 0.07%, 0.06%, 0.05%, 0.04%, 0.03%, 0.02% or 0.01%, of the total weight of the formulation, including the end points. In some embodiments, the formulations of the disclosure, comprise potassium bitartrate, in an amount of less than 0.5% of the total weight of the formulation.

In some embodiments, the formulations of the disclosure, comprise a pH modulating agent or buffer that is sodium hydroxide. In some embodiments, the formulations of the disclosure, comprise sodium hydroxide, in an amount of less than 1%, 0.9%, 0.8%, 0.7%, 0.6%, 0.5%, 0.4%, 0.3%, 0.2% or 0.1%, of the total weight of the formulation, including the end points. In some embodiments, the formulations of the disclosure, comprise sodium hydroxide, in an amount of less than 1% of the total weight of the formulation.

In some embodiments, the formulations of the disclosure, comprise a combination of pH modulating agents or buffers that an amount of lactic acid of 1% to 5% by weight of the formulation, an amount of citric acid monohydrate of 1% to 5% by weight of the formulation, an amount of potassium sodium tartrate of 0.1% to 0.5% by weight of the formulation, and an amount of sodium hydroxide of 0.1% to 0.5% by weight of the formulation. In some embodiments, the formulations of the disclosure, comprise a combination of pH modulating agents or buffers that an amount of lactic acid of 1% to 5% by weight of the formulation, an amount of citric acid monohydrate of 1% to 5% by weight of the formulation, an amount of potassium bitartrate of 0.1% to 0.5% by weight of the formulation, and an amount of sodium hydroxide of 0.1% to 0.5% by weight of the formulation.

In some embodiments, the formulations of the disclosure, comprise lactic acid, citric acid monohydrate, potassium bitartrate and sodium hydroxide, in an amount or percentage by weight, as depicted Tables 1 and 2.

In some embodiments, the formulations of the disclosure, comprise a viscosity modulator, wherein the viscosity modulator is a viscosity enhancer. In some embodiments, the viscosity enhancer comprises one or more of hydroxyethyl cellulose, alginic acid, polycarbophil and carbopol. In some embodiments, the formulations of the disclosure, comprise an amount of a viscosity enhancer of between 1% and 10% of the total weight of the formulation, inclusive of the endpoints. In some embodiments, the formulations of the disclosure, comprise an amount of a viscosity enhancer of between 1% and 5% of the total weight of the formulation, inclusive of the endpoints. In some embodiments, the formulations of the disclosure, comprise an amount of a viscosity enhancer of less than 5% of the total weight of the formulation. In some embodiments, the formulations of the disclosure, comprise an amount of a viscosity enhancer of 3% of the total weight of the formulation. In some embodiments, the formulation of the disclosure, the viscosity enhancer comprises hydroxyethyl cellulose.

In some embodiments, formulation of the disclosure, comprise a viscosity enhancer selected from the group consisting of agar, alamic acid, alginic acid, aluminum monostearate, attapulgite, activated, attapulgite colloidal activated, bentonite, bentonite, purified, bentonite magma, carbomer 910, carbomer 934, carbomer 934p, carbomer 940, carbomer 941, carbomer 1342, carbomer copolymer, carbomer homopolymer, carbomer interpolymer, carbopol, carboxymethylcellulose calcium, carboxymethylcellulose sodium, carboxymethylcellulose sodium 12, carrageenan, cellulose, dextrin, gelatin, gellan gum, guar gum, hydroxyethyl cellulose, hydroxypropyl cellulose, hypromellose (formerly hydroxypropyl methylcellulose), magnesium aluminum silicate, maltodextrin, methylcellulose, microcrystalline cellulose, pectin, polyethylene oxide, polyvinyl alcohol, povidone, propylene glycol alginate, polycarbophil, silicon dioxide, silicon dioxide, colloidal sodium alginate, starch corn, starch potato, starch tapioca, starch wheat, tragacanth and xanthan gum, or a combination thereof.

In some embodiments, the formulations of the disclosure, comprise hydroxyethyl cellulose in an amount of 1% to 10% of the total amount of the formulation. In some embodiments, the formulation comprises hydroxyethyl cellulose in an amount of 1%, 2%, 3%, 4%, 5%, 6%, 7%, 8%, 9% or 9% of the total amount of the formulation. In some embodiments, the formulations of the disclosure, comprise hydroxyethyl cellulose in an amount of 1% to 5% of the total amount of the formulation. In some embodiments, the formulations of the disclosure, comprise hydroxyethyl cellulose in an amount of 3% of the total weight of the formulation.

In some embodiments, the formulations of the disclosure, comprise alginic acid in an amount of less than 5%, less than 4%, less than 3%, less than 2% or less than 1% of the total weight of the formulation. In some embodiments, the formulations of the disclosure, comprise alginic acid in an amount of 4%, 3%, 2% or 1% of the total weight of the formulation. In some embodiments, the formulations of the disclosure, comprise alginic acid in an amount of less than 5% of the total weight of the formulation.

In some embodiments, the formulations of the disclosure, comprise polycarbophil in an amount of less than 5%, less than 4%, less than 3%, less than 2% or less than 1% of the total weight of the formulation. In some embodiments, the formulations of the disclosure, comprise polycarbophil in an amount of 4%, 3%, 2% or 1% of the total weight of the formulation. In some embodiments, the formulations of the disclosure, comprise polycarbophil in an amount of less than 5% of the total weight of the formulation.

In some embodiments, the formulations of the disclosure, comprise carbopol in an amount of less than 5%, less than 4%, less than 3%, less than 2% or less than 1% of the total weight of the formulation. In some embodiments, the formulations of the disclosure, comprise carbopol in an amount of 4%, 3%, 2% or 1% of the total weight of the formulation. In some embodiments, the formulations of the disclosure, comprise carbopol in an amount of less than 5% of the total weight of the formulation.

In some embodiments, the formulations of the disclosure, comprise hydroxyethyl cellulose in an amount of 1% to 5%; alginic acid in an amount of less than 5%; polycarbophil in an amount of less than 5%; and carbopol in an amount of less than 5%, of the total weight of the formulation.

In some embodiments, the formulation comprises an amount of a viscosity enhancer less than 1% of the total weight of the formulation. In some embodiments, the formulation comprises an amount of a viscosity enhancer of 0.1% to 1% of the total weight of the formulation. In some embodiments, the formulation comprises an amount of a viscosity enhancer of 0.1% to 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.9% to 1% of the total weight of the formulation. In some embodiments, the formulation comprises an amount of a viscosity enhancer of about 0.5% of the total weight of the formulation. In some embodiments, the formulation comprises an amount of a viscosity enhancer of 0.5% of the total weight of the formulation. In some embodiments, the viscosity enhancer comprises carbopol. In some embodiments, the carbopol is carbopol-980.

In some embodiments, the formulations of the disclosure, comprise viscosity enhancers hydroxyethyl cellulose, alginic acid, polycarbophil and carbopol in an amount or percentage by weight, as depicted Tables 1 and 2.

In some embodiments, the formulations of the disclosure, comprise a solvent or dispersion medium in an amount of between 10% to 90% of the total weight of the formulation, including the endpoints. In some embodiments, the formulation of the disclosure, comprise a solvent or dispersion medium that is water, ethanol, polyol (for example, glycerol, propylene glycol, and liquid polyethylene glycol, and the like), and suitable mixtures thereof. In some embodiments, the formulation of the disclosure, comprise a solvent or dispersion medium that is water. In some embodiments, the formulations of the disclosure, comprise water in an amount of between 10% and 90%.

In some embodiments, the formulations of the disclosure, comprise water in an amount of between 10% and 80%, 10% and 70%, 10% and 60%, 10% and 50%, 10% and 40%, 10% and 30%, and 10% and 20%, including the end points. In some embodiments, the formulations of the disclosure, comprise water in an amount of 10%, 20%, 30%, 40%, 50%, 60%, 70%, 80% and 90%, of the total weight of the formulation.

Niclosamide is naturally soluble in alcohol. However, the presence of high amounts of alcohols e.g., ethanol, in formulations known in the art for intravaginal or intrarectal administration, can cause several adverse effects including dryness, destruction of natural flora at site of administration and other issues. The niclosamide formulations of the present disclosure, comprise niclosamide dissolved in non-alcoholic solvents.

In some embodiments, the formulations of the disclosure are substantially free of alcohol (e.g., ethanol) or comprise alcohol(s) at a concentration of less that 1% of the total weight of the formulation. In some embodiments, the formulations of the disclosure comprise alcohol at a concentration of less than about 0.9%, less than about 0.8%, less than about 0.7%, less than about 0.6%, less than about 0.5%, less than about 0.4%, less than about 0.3%, less than about 0.2% or less than about 0.1% of the total weight of the formulation. In some embodiments, the formulations of the disclosure comprise alcohol at a concentration of less 0.9%, less 0.8%, less 0.7%, less 0.6%, less 0.5%, less 0.4%, less 0.3%, less 0.2% or less 0.1% of the total weight of the formulation. In some embodiments, the formulations of the disclosure comprise one or more alcohol at a concertation of about 0.2% of the total weight of the formulation. In some embodiments, the formulations of the disclosure comprise one or more alcohol at a concentration of 0.2% of the total weight of the formulation. In some embodiments, the formulations of the disclosure comprise benzyl alcohol at a concentration of 0.2% of the total weight of the formulation. In some embodiments, the alcohol can be ethyl alcohol, methyl alcohol, propyl alcohol, butyl alcohol, benzyl alcohol or a combination thereof. In some embodiments, the formulations of the disclosure do not comprises of any amount or concentration or is essentially free of an alcohol selected from ethyl alcohol, methyl alcohol, propyl alcohol or butyl alcohol.

In some embodiments, formulations of the disclosure, including a semi-solid form, a gel, a lotion or a cream, comprise water in an amount or percentage by weight, as depicted in Tables 1 and 2.

In some embodiments, the formulations of the disclosure, comprise a preservative. In some embodiments, the preservative comprises one or more of benzyl alcohol, chlorhexidine gluconate and benzoic acid. In some embodiments of the formulations of the disclosure, the formulation comprises an amount of the preservative of less than 1% of the total weight of the formulation. In some embodiments, the formulation comprises an amount of the preservative of about 0.2% of the total weight of the formulation. In some embodiments, the formulation comprises an amount of the preservative of 0.2% of the total weight of the formulation. In some embodiments, the formulation comprises an amount of the preservative of less than 0.2% of the total weight of the formulation. In some embodiments, the formulations of the disclosure, comprise an amount of preservative between 0.1% o 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.95 to 0.99%, of the total weight of the formulation, including the end points. In some embodiments, the preservative comprises benzyl alcohol or benzoic acid. In some embodiments, the preservative comprises benzyl alcohol. In some embodiments, the preservative comprises benzoic acid.

In some embodiments, the formulations of the disclosure, comprise an amount of the preservative of less than 1% of the total weight of the formulation. In some embodiments, the preservative comprises benzyl alcohol. In some embodiments, the formulations of the disclosure, comprise an amount of the preservative of less than 0.2% of the total weight of the formulation. In some embodiments, the preservative comprises benzyl alcohol or benzoic acid. In some embodiments, the formulations of the disclosure, comprise an amount of the preservative of less than 0.12% of the total weight of the formulation. In some embodiments, the preservative comprises chlorhexidine gluconate.

In some embodiments, the formulations of the disclosure, comprise a preservative that is any one or more of Vitamin A, Vitamin C, Vitamin E, retinyl palmitate, methionine, BHA (butylatedhydroxyanisole), BHT (butylatedhydroxytoulene), selenium, cysteine propyl gallate, phenol, parabens including but not limited to ethyl paraben, methyl paraben, propyl paraben, butyl paraben, EDTA, citric acid, sodium citrate, benzyl alcohol, chlorobutanol, meta cresol, chloro cresol, benzoic acid, sorbic acid, thiomersal, bronopol diols, propylene glycol, benzylkonium chloride, benzethonium chloride, chlorhexidine gluconate and benzoic acid. In some embodiments, the formulations of the disclosure, comprise a preservative that is a combination of benzyl alcohol, chlorhexidine gluconate and benzoic acid. In some embodiments, the formulations of the disclosure, comprise a preservative in an amount of less than 0.12% to less than 1% of the total weight of the formulation.

In some embodiments, the formulations of the disclosure, comprise benzyl alcohol in an amount of between less than 0.12% to less than 1% of the total weight of the formulation. In some embodiments, the formulations of the disclosure, comprise benzyl alcohol in an amount of less than 1% of the total weight of the formulation. In some embodiments, the formulations of the disclosure, comprise benzyl alcohol in an amount of between less than 1%, 0.5%, 0.4%, 0.3% and 0.2%, of the total weight of the formulation, including the end points.

In some embodiments, the formulations of the disclosure, comprise chlorhexidine gluconate in an amount of between less than 0.12% to less than 1% of the total weight of the formulation. In some embodiments, the formulations of the disclosure, comprise chlorhexidine gluconate in an amount of less than 0.12% of the total weight of the formulation. In some embodiments, the formulations of the disclosure, comprise chlorhexidine gluconate in an amount of between less than 0.12%, 0.11%, 0.10%, 0.09%, 0.08%, 0.07%, 0.06%, 0.05%, 0.04%, 0.03% and 0.02%, of the total weight of the formulation, including the end points.

In some embodiments, the formulations of the disclosure, comprise benzoic acid in an amount of between less than 0.12% to less than 1% of the total weight of the formulation. In some embodiments, the formulations of the disclosure, comprise benzoic acid in an amount of less than 0.2% of the total weight of the formulation. In some embodiments, the formulations of the disclosure, comprise benzoic acid in an amount of between less than 0.2%, 0.1%, 0.09%, 0.08%, 0.07%, 0.06%, 0.05%, 0.04%, 0.03% and 0.02%, of the total weight of the formulation, including the end points.

In some embodiments, the formulations of the disclosure, comprise benzyl alcohol in an amount of between 0.1% o 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.95 to 0.99%, of the total weight of the formulation, including the end points. In some embodiments, the formulations of the disclosure, comprise benzoic acid in an amount of between 0.1% o 0.2%, 0.2% to 0.3%, 0.3% to 0.4%, 0.4% to 0.5%, 0.5% to 0.6%, 0.6% to 0.7%, 0.7% to 0.8%, 0.8% to 0.9% or 0.95 to 0.99%, of the total weight of the formulation, including the end points.

In some embodiments, the formulation of the disclosure, comprises benzyl alcohol in an amount of less than 1%; chlorhexidine gluconate in an amount of less than 0.12%; and benzoic acid in an amount of less than 0.2% of the total weight of the formulation. In some embodiments, the formulation of the disclosure, comprise benzyl alcohol, chlorhexidine gluconate and benzoic acid in an amount or percentage weight as depicted in Table 1.

TABLE 1
Ingredient list with function and purpose, of
exemplary formulation comprising niclosamide.

| Ingredient | Function | Purpose | Amount (w/w) |
|---|---|---|---|
| niclosamide | API | inhibits sperm motility | Minimum dose to see |
| | | | effect is: 0.13% (4 mM) |
| | | | Most likely formulation: |
| | | | 1-5% (100 mM = 3.27%) |
| | | | Max: 10% |
| PEG-400 | excipient | humectant, lubricant, | 0-65% |
| | | solvent | |
| sodium chloride | excipient | adjusting osmolality | <1% |
| lactic acid | excipient | pH modulation | <10% |
| citric acid monohydrate | excipient | pH modulation | <5% |
| potassium bitartrate | excipient | pH modulation | <0.5% |
| sodium hydroxide | excipient | pH modulation | <1% |
| hydroxyethyl cellulose | excipient | viscosity enhancer | Most likely formulation: |
| | | | 1-5% (3% ideal) Max: 10% |
| alginic acid | excipient | viscosity enhancer | <5% |
| polycarbophil | excipient | viscosity enhancer | <5% |
| carbopol | excipient | viscosity enhancer | <5% |
| water | excipient | solvent | 10-90% |
| benzyl alcohol | excipient | preservative | <1% |
| chlorhexidine gluconate | excipient | preservative | <0.12% |
| benzoic acid | excipient | preservative | <0.2% |
| | | Total: | 100% |

In some embodiments, the formulation of the disclosure, comprise niclosamide, PEG-400, carbopol-980 and benzyl alcohol in an amount in an amount or percentage weight as depicted in Table 2.

TABLE 2
Ingredient list with function and purpose,
of gel formulation comprising niclosamide.

| Ingredient | Function | Purpose | Amount (w/w) |
|---|---|---|---|
| niclosamide | API | inhibits sperm motility | Gel formulation: 3-5% |
| | | | (153 mM = 5%) |
| PEG-400 | excipient | humectant, lubricant, | 35%-65% |
| | | solvent | |
| sodium chloride | excipient | adjusting osmolality | 0.1% to 1% |
| lactic acid | excipient | pH modulation | 1% to 5% |
| citric acid monohydrate | excipient | pH modulation | 1% to 5% |
| potassium sodium tartrate | excipient | pH modulation | 0.1% to 0.5% |
| sodium hydroxide | excipient | pH modulation | 0.1% to 0.5% |
| carbopol | excipient | viscosity enhancer | 0.1% to 1% |
| water | excipient | solvent | 10% to 60% |
| benzyl alcohol | excipient | preservative | 0.1% to 0.5% |
| | | Total: | 100% |

In some embodiments of the formulations of the disclosure, the formulation comprises, a) niclosamide at a concentration of 3% to 5% by weight of the formulation; b) a humectant, a lubricant or a solvent, wherein the humectant, the lubricant or the solvent comprises an amount of PEG-400 of 35% to 65% by weight of the formulation and an amount of water of 10% to 60% by weight of the formulation; c) an osmolality modulator, wherein the osmolality modulator comprises an amount of sodium chloride of 0.1% to 1% by weight of the formulation; d) at least one pH modulator, wherein the at least one pH modulator comprises an amount of lactic acid of 1% to 5% by weight of the formulation, an amount of citric acid monohydrate of 1% to 5% by weight of the formulation, an amount of potassium sodium tartrate of 0.1% to 0.5% by weight of the formulation, and an amount of sodium hydroxide of 0.1% to 0.5% by weight of the formulation; e) at least one viscosity modulator, wherein the at least one viscosity modulator comprises an amount of carbopol of 0.1% to 1% by weight of the formulation; and 0 at least one preservative, wherein the at least one preservative comprises an amount of benzyl alcohol of 0.1% to 0.5% by weight of the formulation.

In some embodiments of the formulations of the disclosure, the formulation comprises, a) niclosamide at a concentration of 3% to 5% by weight of the formulation; b) a humectant, a lubricant or a solvent, wherein the humectant, the lubricant or the solvent comprises an amount of PEG-400 of 35% to 65% by weight of the formulation and an amount of water of 0% to 60% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments of the formulations of the disclosure, the formulation comprises, a) niclosamide at a concentration of 5% by weight of the formulation; b) a humectant, a lubricant or a solvent, wherein the humectant, the lubricant or the solvent comprises an amount of PEG-400 of 65% by weight of the formulation and an amount of water of 22.65% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and 0 at least one preservative, wherein the at least one preservative comprises an amount of benzyl alcohol of 0.2% by weight of the formulation.

In some embodiments of the formulations of the disclosure, the formulation comprises, a) niclosamide at a concentration of 5% by weight of the formulation; b) a humectant, a lubricant or a solvent, wherein the humectant, the lubricant or the solvent comprises an amount of PEG-400 of 35% by weight of the formulation and an amount of water of 52.65% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments of the formulations of the disclosure, the formulation comprises, a) niclosamide at a concentration of 3% by weight of the formulation; b) a humectant, a lubricant or a solvent, wherein the humectant, the lubricant or the solvent comprises an amount of PEG-400 of 65% by weight of the formulation and an amount of water of 24.65% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments of the formulations of the disclosure, the formulation comprises, a) niclosamide at a concentration of 3% by weight of the formulation; b) a humectant, a lubricant or a solvent, wherein the humectant, the lubricant or the solvent comprises an amount of PEG-400 of 35% by weight of the formulation and an amount of water of 54.65% by weight of the formulation; c) an osmolality modulator, wherein the osmolality modulator comprises an amount of sodium chloride of 0.7% by weight of the formulation; d) at least one pH modulator, wherein the at least one pH modulator comprises an amount of lactic acid of 3.5% by weight of the formulation, an amount of citric acid monohydrate of 2% by weight of the formulation, an amount of potassium sodium tartrate of 0.35% by weight of the formulation, and an amount of sodium hydroxide of 0.1% by weight of the formulation; e) at least one viscosity modulator, wherein the at least one viscosity modulator comprises an amount of carbopol of 0.5% by weight of the formulation; and f) at least one preservative, wherein the at least one preservative comprises an amount of benzyl alcohol of 0.2% by weight of the formulation.

In some embodiments, the formulations of the disclosure, comprise: a) niclosamide at a concentration of 100 mM by weight of the formulation: b) a humectant, a lubricant or a solvent, wherein the humectant, the lubricant or the solvent comprises an amount of PEG-400 of between 0.1% and 65% by weight of the formulation and an amount of water of between 10% and 90% by weight of the formulation; c) an osmolality modulator, wherein the osmolality modulator comprises an amount of sodium chloride of less than 1% by weight of the formulation; d) at least one pH modulator, wherein the at least one pH modulator comprises an amount of lactic acid of less than 10% by weight of the formulation, an amount of citric acid monohydrate of less than 5% by weight of the formulation, an amount of potassium bitartrate of less than 0.5% by weight of the formulation, and an amount of sodium hydroxide of less than 1% by weight of the formulation; e) at least one viscosity modulator, wherein the at least one viscosity modulator comprises an amount of hydroxyethyl cellulose of 3% by weight of the formulation, an amount of alginic acid of less than 5% by weight of the formulation, an amount of polycarbophil of less than 5% by weight of the formulation and an amount of carbopol of less than 5% by weight of the formulation; and f) at least one preservative, wherein the at least one preservative comprises an amount of benzyl alcohol of less than 1% by weight of the formulation, an amount of chlorhexidine gluconate of less than 0.12% by weight of the formulation, and an amount of benzoic acid of less than 0.2% by weight of the formulation.

In some embodiments, the formulation including those in which the formulations of the disclosure, including a semi-solid form, a gel, a lotion or a cream, comprise the ingredients as depicted in Tables 1 and 2 in the amount or percentage by weight, as depicted in Tables 1 and 2.

In some embodiments, the formulation of the disclosure, can be provided in an amount of between 0.1 mL and 1 mL, 0.1 mL and 0.5 mL, 0.5 mL and 1 mL, 1 mL and 1.5 mL, 1.5 mL and 2 mL, 2 mL and 3 mL, 3 mL and 4 mL, 4 mL and 5 mL, 5 mL and 6 mL, 6 mL and 7 mL, 7 mL and 8 mL, 8 mL and 9 mL, 9 mL and 10 mL, 10 mL and 20 mL, 20 mL and 30 mL, 30 mL and 40 mL, 40 mL and 50 mL, 50 mL and 60 mL, 60 mL and 70 mL, 70 mL and 80 mL, 80 mL and 90 mL, 90 mL and 100 mL, 100 mL and 200 mL, 200 mL and 300 mL, 400 mL and 500 mL, 500 mL and 600 mL, 600 mL and 700 mL, 800 mL and 900 mL, and 900 mL and 1000 mL, inclusive of the endpoints. In some embodiments, the formulation of the disclosure, can be provided in an amount of between 0.5 mL and 10 mL, inclusive of the endpoints.

In some embodiments, the formulations of the disclosure, are suitable for intravaginal application by an individual who is not a medical professional. In some embodiments, the formulations of the disclosure are suitable for topical administration by the subject. In some embodiments, the formulations of the disclosure are suitable for direct intravaginal application. In some embodiments, the formulations of the disclosure are suitable for direct intrarectal application.

In some embodiments, the formulations of the disclosure may be administered less than 1 hour before sexual activity. In some embodiments, the formulations of the disclosure may be administered less than 1 minute (min), 2 min, 3 min, 4 min, 5 min, 10 min, 15 min, 20 min, 25 min, 30 min, 35 min, 40 min, 45 min, 50 min, 55 min, 60 min or any number of minutes in between, prior to sexual activity. In some embodiments of the methods of treatment of the disclosure, the female subject is healthy. In some embodiments of the methods of treatment of the disclosure, the semen sample is from a healthy male.

In some embodiments of the methods of treatment of the disclosure, the formulation contacts a sperm cell in vivo or in vitro.

In some embodiments, the formulations of the disclosure are pharmaceutical formulations.

In some embodiments, formulations of the disclosure comprise a semi-solid form. In some embodiments, the semi-solid form comprise one or more of a gel, a hydrogel, a dehydrated gel, a hydrated gel, a paste, an ointment, a cream, and a lotion.

Gels of the disclosure include, but are not limited to, controlled release gels, organogels, extended release gels, amphiphilic gels, hydrophilic gels, non aqueous gels, bioadhesive gels, thermosensitive sol-gel reversible hydrogels, complexation gels and hydrogel.

### Sperm Motility Assessment

Sperm motility is a functional measurement of the sperm themselves. Sperm may be sampled either directly from semen or from washed sperm samples, and assessed for motility either manually or by CASA (Computer Assisted Sperm Analysis) (Chapter 59—Male Reproductive System. Systems Toxicologic Pathology. Dianne M. Creasy, Robert E. Chapin, in Haschek and Rousseaux's Handbook of Toxicologic Pathology (Third Edition), 2013; Amann R P et al. Computer-assisted sperm analysis (CASA): capabilities and potential developments. Theriogenology. 2014 Jan. 1; 81(1):5-17). Sperm motility (expressed as %) may be evaluated in duplicate within 1 h of semen collection, by counting moving and nonmoving sperm in several microscopic fields. In some embodiments, at least 200 spermatozoa are counted and classified as (1) “rapidly progressive” (class A) that move forward with speed of at least 25 μm/s (half a tail or 5 head lengths); (2) “slowly progressive” (class B) that move forward with more than 5 μm/s (one head lengths) but less than 25 μm/s; (3) “nonprogressive” (class C) that are slow and only move less than 5 μm/s; and (4) “immotile” (class D) that do not move and appear dead. In preferred embodiments, only complete spermatozoa (head with tail) are included in such counts. Slow-moving sperm (class B+C) can be easily and accurately counted compared with rapidly moving (class A) sperm. A multi-button tally, preferably a digital one, is used to differentially count such motility (for additional detail, see Chapter 23: Standardized semen analysis and quality control management for multicenter male reproductive toxicology clinical trials, Sikka S. C. et al., in Bioenvironmental Issues Affecting Men's Reproductive and Sexual Health, 2018, the contents of which are incorporated herein by reference in their entirety).

### Sperm Viability Assessment

Sperm viability as described herein is defined as the percentage of the total number of sperm cells in a semen sample that are alive. In some embodiments, the sperm viability can be determined by the percentage of motile sperms in a semen sample. In some embodiments, the sperm viability can be determined by the percentage of sperm cells with an intact cell membrane in a semen sample. The percentage of sperm cells with an intact cell membrane in a semen sample can be determines by a staining procedure using a dye, wherein the live sperms can extrude out the dye and the dead sperms retain the dye. In some embodiments, the percentage of sperm cells with an intact cell membrane in a semen sample can be determines by eosin-nigrosin staining. In some embodiments, sperm viability of a semen sample is considered low if less that 25%, less than 30% or less than 40% of the total number of sperms in the semen sample are viable. In some embodiments, the the sperm viability can be determined using any standard procedures known in the art, for example WHO Laboratory manual for examination of human semen and sperm-cervical mucus interaction. 5th ed. Geneva. World Health Organization. Switzerland; 2010.

### Sperm Metabolism Assessment

Sperm metabolism as described herein is defined measuring the level of energy metabolism in sperm cells in a semen sample in terms of generation of andenosine triphosphate (ATP) using oxydative phosphorylation in mitochondria and/or glycolysis in cytoplasm. In some embodiments, the level of energy metabolism in sperm cells in a semen sample can be measured in terms of the level of ATP in a semen sample using a bioluminiscent assay, using any standard procedure known in the art (Vermeulen L, Comhaire F: Detection of cytotoxic sperm antibodies using ATP determination: a comparison with other immunological methods. In Luminescent Assays: Perspectives in Endocrinology and Clinical Chemistry, Edited by M Serio, M Pazzagli. New York, Raven Press, 1982, p 89).

### Assessment of Probability of Pregnancy

Pregnancy assessment or the determination of probability of pregnancy as described herein, can be determined by the number of female subjects who become pregnant or conceive after one or more recorded sexual intercourse or coitus, in a group of female subject all of whom have been contacted with the formulation of the disclosure intravaginally, as compared to a group of female subjects, that have not been contacted with the formulation of the disclosure intravaginally. In some embodiments, the the determination of probability of pregnancy as described herein, can be determined by the number of times a female subject who has been contacted with the formulation of the disclosure, becomes pregnant or conceive after one or more recorded sexual intercourse or coitus, as compared to a female subjects, that has not been contacted with the formulation of the disclosure intravaginally. The detection of pregnancy or conceiving as described herein, is defined as detection of implantation of an embryo in the uterus of the female subject. In some embodiments, the detection of implantation of an embryo in the uterus of the female subject, can be determined by blood test (e.g. for human chorionic gonadotropin (HCG), estriol and/or inhibin levels), vaginal biopsy, ultrasound or a combination thereof.

### Therapeutically Effective Amounts

A therapeutically effective dose may be a therapeutically effective amount of the formulation of the disclosure, that when contacted with a sperm cell, is sufficient to decrease motility of that cell when compared with a control value. A therapeutically effective dose may be a portion of a therapeutically effective amount. For example, a therapeutically effective dose may be half of the therapeutically effective amount provided in two separate administrations over a period of time, that in aggregate, provide the therapeutically effective amount of the formulation prior to the sexual activity.

A therapeutically effective amount of the formulation of the disclosure relates generally to the amount needed to achieve a therapeutic objective. A “therapeutically effective amount” of the formulation of the disclosure is an amount of the formulation that when contacted with one or more sperm cells, for a sufficient amount of time, induces a decrease or an inhibition of the motility of one or more sperm cell(s), as compared to a control value (e.g. the motility of the sperm cells in a semen sample that has not been contacted with the formulation).

A “sufficient amount of time” of contacting the formulation of the disclosure with a semen sample relates generally to the amount needed to achieve a therapeutic objective. “Sufficient amount of time” of the formulation of the disclosure is an amount of time for which when the formulation, when contacted, with one or more sperm cell(s) induces a decrease or an inhibition of the motility of at least one of the one or more sperm cells.

In some embodiments the decrease or inhibition of the motility of the sperm cell is measured as a percentage reduction in progression speed of the sperm cell. In some embodiments the decrease or inhibition in the motility of the sperm cell is measured as a percentage reduction in linear progression speed of the sperm cell relative to a control value.

In some embodiments, the control value is the speed of a healthy sperm cell or an average speed of a plurality of healthy sperm cells. In some embodiments, the healthy sperm cell(s) have not contacted the formulation.

In some embodiments the therapeutically effective amount of the formulation of the disclosure is an amount that when contacted with one or more sperm cell(s) induces a reduction in sperm motility by at least 50%, 60%, 70%, 80%, 90% or 99% or any percentage in between, in the motility of the one or more sperm cell(s) relative to the control value. In some embodiments the therapeutically effective amount of the formulation of the disclosure is an amount that when contacted with one or more sperm cells induces a complete reduction in number of motile sperm cells (zero motile sperm or 100% immobile sperm cells), for example, in an in vitro test, relative to the number of motile sperm cells as measured before contact with the formulation, or relative to the number of motile sperm cells as measured in a sample that has not contacted the formulation.

In some embodiments a therapeutically effective amount of the formulation of the disclosure is an amount that decreases or reduces the motility of the sperm cell or a portion of the plurality of sperm cells from a speed of more than 25 μm/s (one head lengths), before contacting with the formulation, to a speed of less than 5 μm/s (one head lengths), after contacting with the formulation. In some embodiments a therapeutically effective amount of the formulation of the disclosure is an amount that decreases or reduces the motility of the sperm cell or a portion of the plurality of sperm cells from a speed of more than 25 μm/s (one head lengths), before contacting with the formulation, to a speed of 0 μm/s (one head lengths), after contacting with the formulation. In some embodiments a therapeutically effective amount of the formulation of the disclosure is an amount that decreases or reduces the motility of the sperm cell or a portion of the plurality of sperm cells from a speed of at least 25 μm/s (half a tail or 5 head lengths), before contacting with the formulation to less than 25 μm/s to a speed of after contacting with the formulation. In some embodiments a therapeutically effective amount of the formulation of the disclosure is an amount that decreases or reduces the motility of the sperm cell or a portion of the plurality of sperm cells from a speed of at least 5 μm/s (head lengths), before contacting with the formulation to less than 25 μm/s to a speed of after contacting with the formulation.

In some embodiments the therapeutically effective amount of the formulation of the disclosure is an amount that contacted with a plurality of sperm cells induces an decrease in motility of at least 90%, 95%, 97%, 99%, 100% or any percentage in between of the plurality of sperm cells of sperm cells from at least 25 μm/s (half a tail or 5 head lengths) before contacting with the formulation, to a speed of less than 5 μm/s after contacting with the formulation.

In some embodiments the therapeutically effective amount of the formulation of the disclosure is an amount that contacted with a plurality of sperm cells induces an decreases or reduces the motility of at least 90%, 95%, 97%, 99%, 100% or any percentage in between of the plurality of sperm cells from at least 25 μm/s (half a tail or 5 head lengths) before contacting with the formulation, to a speed of 0 μm/s after contacting with the formulation.

In some embodiments, the therapeutically effective amount of the formulation of the disclosure is an amount is an amount that inhibits or downregulates sperm viability by about 60%, 65%, 70%, 75%, 80%, 85%, 90%, 95%, 100%, or any percentage in between, in comparison to a control value. In some embodiments, the control value is a predetermined value. In some embodiments, the control value is an average value of sperm viability measured from healthy sperm cells obtained from one or more individual donors. In some embodiments, the healthy sperm cells have not contacted the formulation. In some embodiments, the control value is determined in vitro.

In some embodiments, the therapeutically effective amount of the formulation of the disclosure is an amount is an amount that inhibits or downregulates sperm metabolism as measured by the level of ATP in a semen sample by about 60%, 65%, 70%, 75%, 80%, 85%, 90%, 95%, 100%, or any percentage in between, in comparison, to a control value. In some embodiments, the control value is a predetermined value. In some embodiments, the control value is an average value of sperm metabolism as measured by the level of ATP in healthy semen samples obtained from one or more individual donors. In some embodiments, the healthy semen samples have not contacted the formulation. In some embodiments, the control value is determined in vitro.

In some embodiments, the therapeutically effective amount of the formulation of the disclosure is an amount that when contacted intravaginally to a female subject participating in sexual intercourse or coitus, before the sexual intercourse or coitus prevents or reduces the probability of conception, resulting from the sexual intercourse, activity or coitus, as compared to a female subject to whom the formulation of the disclosure has not been contacted intravaginally. In some embodiments, the therapeutically effective amount of the formulations of the disclosure, is an amount that reduces the the probability of conception in a female subject contacted with the formulation by at least 50%, at least 60%, at least 70%, at least 80%, at least 90%, a compared to a female subject to whom the formulation of the disclosure has not been contacted intravaginally. In some embodiments, the therapeutically effective amount of the formulations of the disclosure, is an amount that reduces the the probability of conception in a female subject contacted with the formulation by 100%, a compared to a female subject to whom the formulation of the disclosure has not been contacted intravaginally.

In some embodiments, including those in which the formulations of the disclosure comprise a semi-solid or a liquid form, the formulation may comprise a concentration of the niclosamide, at a concentration between 1mM and 100 mM, inclusive of the endpoints. In some embodiments, the formulation may comprise a concentration of the niclosamide, at a concentration between 1 mM and 10 mM, 10 mM and 20 mM, 20 mM and 30 mM, 30 mM and 40 mM, 40 mM and 50 mM, 50 mM and 60 mM, 60 mM and 70 mM, 70 mM and 80 mM, 80 mM and 90 mM, 90 mM and 100 mM, inclusive of the endpoints. In some embodiments, the formulation may comprise a concentration of niclosamide at a concentration of 10 mM. In some embodiments, the formulation may comprise niclosamide at a concentration of 50 mM. In some embodiments, the formulation may comprise niclosamide at a concentration of 100 mM.

Provided herein is a method of decreasing the probability of conception in a female subject in need thereof, comprising contacting the female subject with an effective amount of the formulation of the disclosure intravaginally prior to a sexual intercourse or coitus, wherein the probability of conceiving post the sexual intercourse or coitus of the female subject contacted with the formulation, is less than the probability of conceiving post the sexual intercourse or coitus of the female subject that has not been contacted with the formulation. In some embodiments, the probability of conceiving of a female subject who has been contacted with the effective amount of the formulation of the disclosure intravaginally prior to a sexual intercourse or coitus, is at least 50%, at least 60%, at least 70%, at least 80%, at least 90%, at least 95% or at least 99% less than the probability of conceiving of a female subject who has not been contacted with the formulation.

Provided herein is a method of making a niclosamide formulation, wherein the method comprises: a) combining an amount of niclosamide with a amount of PEG-400 in a first vessel to form a solution; b) combining an amount of benzoic acid and/or benzyl alcohol with the solution of (a) to form a PEG fraction; c) combining an amount of water, an amount of sodium chloride, an amount of citric acid, an amount of lactic acid, an amount of potassium tartrate and an amount of carbopol 980 in a separate vessel to form an aqueous fraction; d) combining the aqueous fraction of (c) with the PEG fraction of (b); e) adding an amount of sodium hydroxide to the mixture of (d) to adjust the pH; f) optionally adding water to the mixture of (e); and g) mixing the mixture of (e) or (f) till a homogenous mixture is obtained. In some embodiments of the method of manufacturing the formulations of the disclosure, the step (a) comprises mixing by stirring for about 30 minutes at a speed of between 500-600 rotations per minute (RPM). In some embodiments of the method of manufacturing the formulations of the disclosure, the step (a) further comprises stirring the mixture with simultaneously heating and stirring. In some embodiments of the method of manufacturing the formulations of the disclosure, the heating of the mixture is done to a temperature of about 65° C. In some embodiments of the method of manufacturing the formulations of the disclosure, the step (c) comprises mixing by stirring for about 15 minutes at a speed of between 100-200 RPM. In some embodiments, the step (c) comprises mixing for a sufficient amount of time to allow hydration of the carbopol 980. In some embodiments of the method of manufacturing the formulations of the disclosure, the step (d) comprises mixing by stirring for about 15 minutes at a speed of between 900-1200 RPM. In some embodiments of the method of manufacturing the formulations of the disclosure, the step (g) comprises mixing by stirring for about 10 minutes at a speed of between 500-600 RPM.

Every document cited herein, including any cross referenced or related patent or application, is hereby incorporated herein by reference in its entirety unless expressly excluded or limited. The citation of any document is not an admission that it is prior art with respect to any invention disclosed or claimed herein or that it alone, or in any combination with any some reference or references, teaches, suggests or discloses any such invention. Further, to the extent that any meaning or definition of a term in this document conflicts with any meaning or definition of the same term in a document incorporated by reference, the meaning or definition assigned to that term in this document shall govern. As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

While particular embodiments of the disclosure have been illustrated and described, various some changes and modifications can be made without departing from the spirit and scope of the disclosure. The scope of the appended claims includes all such changes and modifications that are within the scope of this disclosure.

### EXAMPLES

In order that the invention disclosed herein may be more efficiently understood, examples are provided below. It should be understood that these examples are for illustrative purposes only and are not to be construed as limiting the invention in any manner.

### Example 1

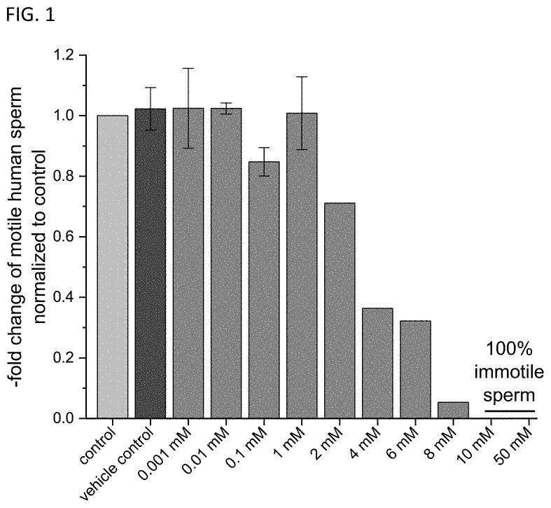

To determine the optimal concentration or dose of niclosamide that completely inhibits the motility of sperm cells in a semen sample (100% inhibition), fresh semen samples were mixed with the formulation in gel form of the disclosure, for 2 minutes. After 2 minutes of incubation with the formulation, the semen samples were collected and analyzed for sperm motility. The fold change in total number of motile sperm cells treated with each concentration of niclosamide, relative to sperm motility normalized to the total number of motile sperm in a control sample (un-treated sample). As for effective concentrations, this in vitro data suggests that the effective niclosamide concentration that causes 100% fold decrease in number of motile sperm cells in a semen sample as compared to a control sample, is 10 mM or higher. Attached is a graph in FIG. 1 that shows the dose-dependent effect of niclosamide on human sperm motility. Data were collected within 2 minutes after mixing the formulation in gel form, with fresh semen, so the effects are instantaneous. As for timing, the gel may be applied 15-20 minutes prior to intercourse to allow gelling to fully occur. Furthermore, the gel may not be efficacious after residing in the vagina for longer than 1 hour.

### Example 2

The objective of the study described herein was to develop a vaginal gel formulation of niclosamide for local administration, which is non-sticky, non-irritating with optimum rheological properties that enables easy extrusion from the dosage form and enables easy application and spreading. As the drug is intended for local action, formulation was designed to ensure minimal absorption into systemic circulation. A dense network of blood vessels is present in vagina making it an excellent route of administration for local drug delivery. Physico-chemical properties such as molecular weight, lipophilicity, molecule solubility, ionization and surface charges etc. may have an impact on drug availability at the site of action. Hence, these properties were considered while designing the vaginal gel formulation for optimum clinical effectiveness. The water solubility of niclosamide has been reported to be about 13.32 μg/mL in its anhydrous form, but this falls to about 0.61 or 0.96 μg/mL for its monohydrate forms (Alhalaweh, A et al., Mol. Pharm. 2014, 11, 3123-3132; Sanphui, P et al., Cryst. Growth Des. 2012, 12, 4588-4599; van Tonder, E. C et al., Int. J. Pharm. 2004, 269, 417-432). The poor solubility of niclosamide in water and the need to disperse niclosamide within a gel to make a homogenous composition, are some of the major challenges for developing a gel formulation. The study described herein, discloses the development of a niclosamide gel formulation comprising excipients that enable both the solubility of therapeutically effective amounts of niclosamide in the gel formulation, as well as release of a therapeutically effective of the niclosamide from the formulation. Several, solvent/cosolvent systems were screened to identify specific solvents or solvent combinations for. The study described herein, provides details of the screening done to select the most suitable excipients (solvent/cosolvent systems, gelling agents, viscosity modulators, buffers/pH modulator and preservatives) and their optimal amounts/concentrations for use in the formulations of the disclosure, based on the compatibility with niclosamide and effect on gelling capacity.

Forced Degradation Data of Drug Substance:

Stress test of niclosamide in neutral, acid, alkaline conditions, forced by temperature effects, oxidation by UV on the dry substance was evaluated by Olon SPA (DMF). The following table details the forced degradation data.

TABLE 3
Summary of forced degradation data
of drug substance (niclosamide).

| S. | | |
|---|---|---|
| No. | Conditions | % Degradation |
| 1 | No stress Conditions | Impurity 3-0.04% |
| 2 | UV Exposure for 7 days | No degradation |
| 3 | Temperature Effect | No degradation |
| | (100° C. for 8 hours) | |
| 4 | Neutral Hydrolysis | Impurity 1-0.02% |
| | (3 mL H₂O at 100° C. for 8 hours) | Impurity 2-0.03% |
| 5 | Acid Hydrolysis | No degradation |
| | (3 mL HCl at 100° C. for 8 hours) | |
| 6 | Alkaline Hydrolysis | Impurity 1 & 2 are |
| | (0.1N NaOH at 100° C. for 2 hours) | formed and potency |
| | | decreased to 97% |
| 7 | Oxidative Hydrolysis | No degradation |
| | (3 mL H₂O₂ at 100° C. for 8 hours) | |

Conclusion: Based on forced degradation data it was concluded that the drug substance is prone to alkaline hydrolysis under increased temperature conditions.

Preformulation Studies.

Solubility studies of drug substance: Solubility of niclosamide was conducted in various aqueous buffers and additionally solubility was conducted using different grades of Polyethylene glycol (PEG).

Saturation Solubility in Buffers:

Solubility was established using 100 mL of each of the chosen buffers, and was conducted using shake flask method. To the chosen buffer, drug substance was added in increments of small amounts until the solution showed resistance to solubilize. Further, the solution was agitated at 37° C. for 24 hours and checked physically for un-dissolved mass. This saturated solution was centrifuged and injected an aliquot in to the chromatograph.

TABLE 4
Results of saturation solubility of niclosamide in various Buffers

| S. No. | Buffer/Solvent | Solubility (mg/mL) |
|---|---|---|
| 1 | pH 1.2 (0.1N HCl) | Nil |
| 2 | pH 2.0 (0.01N HCl) | Nil |
| 3 | pH 3.0 acid phthalate buffer | Nil |
| 4 | pH 4.0 acid phthalate buffer | Nil |
| 5 | pH 4.5 acetate buffer | Nil |
| 6 | pH 5.1 acetate buffer | Nil |
| 7 | water^{§} | 0.005 |

^{§}Solubility assessed at room temperature (~25° C.).

Solubility in Solvents/Co-Solvents:

Solubility of the drug was determined in selected solvent/co-solvents. About 10 g of the solvent was taken and small amount of drug was added in increments till 500 mg. This study was carried out to check if 5% w/w of the drug solubility could be achieved. These solutions were further diluted quantitatively to about test concentration and analyzed using chromatograph.

TABLE 5
Solubility data of niclosamide in solvents

| S. No. | Solvent | Solubility (mg/g) |
|---|---|---|
| 1 | polyethylene glycol (PEG 400) | 47.4 |
| 2 | polyethylene glycol (PEG 1000) | 46.6 |
| 3 | polyethylene glycol (PEG 2000) | 45.6 |

Saturation Solubility in Various Solvents:

Saturation solubility of niclosamide was studied in the solvents, to know the highest concentration of niclosamide that can be solubilized, as depicted in table 6.

TABLE 6
Saturation solubility of niclosamide in solvents

| Quantity | Quantity | | | |
|---|---|---|---|---|
| of Solvent | of API | Procedure | Remarks | Inference |
| 10 g of | *800 mg | 800 mg of API was added gradually | No clear solution, | 700 mg was |
| PEG-400 | | and solubilised by stirring and | few API particles | solubilised |
| | | simultaneous heating at 50° C. for | were observed. | completely. |
| | | 40 min. | | |
| 10 g of | *600 mg | 600 mg of API was added gradually | No clear solution, | 500 mg was |
| PEG-1000 | | and solubilised by continuous stirring | few API particles | solubilised |
| | | and heating at 50° C. for 40 min. | were observed. | completely. |
| 10 g of | *600 mg | 600 mg of API was added gradually | No clear solution, | 500 mg was |
| PEG-2000 | | and solubilised by continuous stirring | few API particles | solubilised |
| | | and heating at 50° C. for 40 min. | were observed. | completely. |

*500 mg of API was added initially; to it 100 mg was added sequentially after complete solubilization of former 100 mg.

Observation: Based on solubility study data, niclosamide was found to be insoluble in studied buffers like 0.1 N HCl, 0.01 N HCl, pH 3.0 acid phthalate buffer, pH 4.0 acid phthalate buffer, pH 4.5 acetate buffer or pH 5.1 acetate buffer. Heating and stirring improved solubilisation of niclosamide in polyethylene glycol. Wherein maximum solubility of 7% w/w in PEG-400 and 5% w/w in PEG-1000 & PEG-2000 was achieved. In further formulation studies, drug substance concentration >7% w/w was desired, so 65° C. of temperature was used for solubilization of API. Hence, a slight impact of heating was observed.

Polymer Screening Studies

Described herein is a study done to shortlist from among several polymers with bio adhesive properties including: polyacrylates (polycarbophil, carbomer, thiolated polyacrylates), cellulosic derivatives (sodium carboxymethylcellulose, hydroxypropylmethylcellulose, hydroxyethylcellulose), based on the gelling capacity in solvents. Polymers used for this study were mixed in the solvents to see their gel formation. All the solutions were kept for 15 hrs at room temperature and observations were made regarding the formation of gel. Table 7 describes the details of solvents with their concentrations, used and it also summarizes the formation of gels.

TABLE 7
Study plan and observations for polymer screening studies.

| | | Polymer | |
|---|---|---|---|
| Sr. | | Concentration | |
| No. | Samples | (% w/w) | Remarks |
| 1 | PEG-400 (5 g) + HEC [Natrosol | 0.5 | Dispersed, no gel formation |
| | 250HHX Pharm] (25 mg) | | |
| 2 | PEG-400 (5 g) + sodium CMC | 0.5 | Dispersed, no gel formation |
| | [Blanose CMC 7HF PH] (25 mg) | | |
| 3 | PEG-400 (5 g) + carbopol 980 NF | 0.5 | Dispersed |
| | (25 mg) | | |
| 4 | PEG-400 (5 g) + HPC [Klucel HF] | 0.5 | Dispersed, no gel formation |
| | (25 mg) | | |
| 5 | PEG-400 (5 g) + pemulen TR-1 NF | 0.5 | Dispersed |
| | (25 mg) | | |
| 6 | PEG-400 (5 g) + polycarbophil | 0.5 | Dispersed |
| | [Noveon AA-1] (25 mg) | | |
| 7 | PEG-400 (5 g) + HPMC K4M | 0.5 | Dispersed |
| | [Methocel K4M] (25 mg) | | |
| 8 | PEG-400 (5 g) + HEC [Natrosol | 0.25 | Dispersed |
| | 250HHX Pharm] (25 mg) + | | |
| | water (5 g) | | |
| 9 | PEG-400 (5 g) + sodium CMC | 0.25 | Dispersed |
| | [Blanose CMC 7HF PH] | | |
| | (25 mg) + water (5 g) | | |
| 10 | PEG-400 (5 g) + carbopol 980 NF | 0.25 | Less viscous gel formed |
| | (25 mg) + water (5 g) | | |
| 11 | PEG-400 (5 g) + HPC [Klucel HF] | 0.25 | Dispersed |
| | (25 mg) + water (5 g) | | |
| 12 | PEG-400 (5 g) + pemulen TR-1 | 0.25 | Less viscous gel formed |
| | NF (25 mg) + water (5 g) | | |
| 13 | PEG-400 (5 g) + polycarbophil | 0.25 | Less viscous gel formed |
| | [Noveon AA-1] (25 mg) + | | |
| | water (5 g) | | |
| 14 | PEG-400 (5 g) + HPMC K4M | 0.25 | Dispersed |
| | [Methocel K4M] (25 mg) + | | |
| | water (5 g) | | |
| 15 | water (20 g) + HEC [Natrosol | 1.0 | Very less viscous gel formed |
| | 250HHX Pharm] (200 mg) | | |
| 16 | water (20 g) + sodium CMC | 1.0 | Viscous gel formed |
| | [Blanose CMC 7HF PH] (200 mg) | | |
| 17 | water (20 g) + carbopol 980 NF | 1.0 | Viscous gel formed |
| | (200 mg) | | |
| 18 | water (20 g) + HPC [Klucel HF] | 1.0 | Very less viscous gel formed |
| | (200 mg) | | |
| 19 | water (20 g) + pemulen TR-1 NF | 1.0 | Viscous gel formed |
| | (200 mg) | | |
| 20 | water (20 g) + polycarbophil | 1.0 | Viscous gel formed |
| | [Noveon AA-1] (200 mg) | | |
| 21 | water (20 g) + HPMC K4M | 1.0 | Very less viscous gel formed |
| | [Methocel K4M] (200 mg) | | |

Observations: Polymers like carbopol, pemulen TR 1, polycarbophil, and HPMC were observed to form gels in PEG-400 alone, and polymers like carbopol, pemulen TR 1, polycarbophil were observed to form gels in mixture of PEG-400 and water.

Drug—excipient compatibility studies: The drug excipient compatibility studies were performed to check the compatibility between the niclosamide API and the proposed inactive ingredients to be used in the formulation. Proposed drug to excipient ratio was selected based on the function of the excipient and maximum concentration to be used. Drug and excipient binary mixtures were prepared by uniform mixing. The blends were packed in clear glass vials and the compatibility was evaluated in closed conditions. In the closed-vial study, the dry mix samples were filled in vials with screw stopper fitting covered with parafilm and stored at 40° C./75% RH, 25° C./60% RH and 2° C.-8° C. storage condition up to 4 weeks. Samples at 40° C./75% RH and 25° C./60% RH were checked for physical description and analyzed for related substances, initially, at 2 weeks and at 4 weeks. Ratio of drug substance to excipients in blend for the compatibility study, are as follows: a) niclosamide:gelling agents/mucoadhesive agents was 1:15; b) niclosamide:solvents/ co-solvents was 1:5; c) niclosamide:pH adjusting agents/neutralizing agents was 1:1; and d) niclosamide:preservatives/anti-oxidants was 1:0.5. Summary of physical description and related substances analysis of binary mixtures of API & excipients at Initial and 40° C./75% RH—4 weeks condition are presented below in Table 8.

TABLE 8
Summary of drug-excipient compatibility study data (physical description and related substances).

| | | | | | | | | | | | | |
|---|---|---|---|---|---|---|---|---|---|---|---|---|
| Sr. | Sample | Combination/ | Stability | Time | | | | | | | Single Max | Total |
| No. | Code | Mixture | Condition | Interval | Description | Imp-1 | Imp-2 | Imp-3 | Imp-4 | Imp-5 | Unk Imp | Impurities |

Active Pharmaceutical Ingredient

| 1 | A | niclosamide | Initial | | Pale yellow | ND | ND | 0.029 | 0.009 | ND | 0.010 | 0.05 |
|---|---|---|---|---|---|---|---|---|---|---|---|---|
| | | | | | color solid | | | | | | (RRT 0.768) | |
| | | | 40° C./75% RH | 4 Weeks | pale yellow | ND | ND | 0.026 | 0.012 | 0.014 | 0.007 | 0.06 |
| | | | | | color solid | | | | | | (RRT 0.768) | |

Gelling Agents/Mucoadhesive Agents

| 2 | B | niclosamide + | Initial | | White color | 0.008 | ND | 0.036 | 0.018 | ND | ND | 0.06 |
|---|---|---|---|---|---|---|---|---|---|---|---|---|
| | | carbopol 980 | | | solid | | | | | | | |
| | | | 40° C./75% RH | 4 Weeks | White color | ND | ND | 0.027 | 0.018 | 0.008 | 0.028 | 0.10 |
| | | | | | solid | | | | | | (RRT 0.126) | |
| 3 | C | niclosamide + | Initial | | light yellow | ND | ND | 0.026 | ND | ND | 0.023 | 0.05 |
| | | hydroxypropyl | | | color solid | | | | | | (RRT 0.113) | |
| | | cellulose | 40° C./75% RH | 4 Weeks | Pale yellow | ND | ND | 0.030 | 0.012 | 0.009 | 0.010 | 0.06 |
| | | (Klucel HF Pharm) | | | color solid | | | | | | (RRT 0.770) | |
| 4 | D | niclosamide + | Initial | | White color | 0.014 | ND | 0.031 | 0.012 | ND | 0.032 | 0.10 |
| | | polycarbophils | | | solid | | | | | | (RRT 0.106) | |
| | | (Noveon AA-1) | 40° C./75% RH | 4 Weeks | off White | ND | ND | 0.029 | 0.017 | 0.009 | 0.007 | 0.07 |
| | | | | | color solid | | | | | | (RRT 0.768) | |
| 5 | E | niclosamide + | Initial | | off-White | 0.004 | ND | 0.034 | 0.013 | ND | 0.006 | 0.06 |
| | | hydroxypropyl | | | color solid | | | | | | (RRT 0.142) | |
| | | methyl cellulose | 40° C./75% RH | 4 Weeks | Pale yellow | ND | ND | 0.027 | 0.011 | 0.009 | 0.007 | 0.05 |
| | | (Methocel K4M) | | | color solid | | | | | | (RRT 0.767) | |
| 6 | F | niclosamide + | Initial | | pale yellow | 0.008 | ND | 0.028 | 0.012 | ND | 0.005 | 0.05 |
| | | hydroxy | | | color solid | | | | | | (RRT 0.164) | |
| | | ethyl cellulose | 40° C./75% RH | 4 Weeks | Yellow color | ND | ND | 0.027 | 0.008 | 0.010 | 0.008 | 0.05 |
| | | (Natrosol 250HHX | | | solid | | | | | | (RRT 0.767) | |
| | | Pharm) | | | | | | | | | | |
| 7 | G | niclosamide + | Initial | | off-White | ND | ND | 0.025 | 0.015 | ND | 0.054 | 0.10 |
| | | sodium | | | color solid | | | | | | (RRT 0.112) | |
| | | carboxy methyl | 40° C./75% RH | 4 Weeks | light pink | ND | 0.019 | 0.019 | 0.006 | 0.008 | 0.027 | 0.09 |
| | | cellulose | | | color solid | | | | | | (RRT 0.623) | |
| | | (Blanose CMC 7HF | | | | | | | | | | |
| | | PH) | | | | | | | | | | |
| 8 | H | niclosamide + | Initial | | off-White | ND | 0.004 | 0.038 | 0.005 | ND | 0.013 | 0.07 |
| | | pemulen TR-1 | | | color solid | | | | | | (RRT 0.126) | |
| | | | 40° C./75% RH | 4 Weeks | off-White | ND | ND | 0.028 | 0.015 | 0.007 | 0.008 | 0.07 |
| | | | | | color solid | | | | | | (RRT 0.768) | |
| 9 | I | niclosamide + | Initial | | pale yellow | ND | ND | 0.034 | ND | ND | 0.006 | 0.04 |
| | | poloxamer | | | color solid | | | | | | (RRT 0.112) | |
| | | (Kolliphor P188) | 40° C./75% RH | 4 Weeks | Pale yellow | ND | ND | 0.026 | 0.011 | 0.006 | 0.008 | 0.05 |
| | | | | | color solid | | | | | | (RRT 0.768) | |

Solvents/Co-solvents

| 10 | J | niclosamide + | Initial | | pale yellow | 0.002 | ND | 0.030 | 0.012 | 0.009 | 0.006 | 0.06 |
|---|---|---|---|---|---|---|---|---|---|---|---|---|
| | | propylene glycol | | | color liquid | | | | | | (RRT 0.761) | |
| | | | 40° C./75% RH | 4 Weeks | pale yellow | ND | ND | 0.027 | 0.011 | 0.010 | 0.007 | 0.06 |
| | | | | | color liquid | | | | | | (RRT 0.767) | |
| 11 | K | niclosamide + | Initial | | pale yellow | 0.002 | ND | 0.028 | 0.012 | 0.007 | 0.006 | 0.06 |
| | | polyethylene | | | color solid | | | | | | (RRT 0.761) | |
| | | glycol 1000 | 40° C./75% RH | 4 Weeks | pale yellow | ND | ND | 0.0026 | 0.013 | 0.008 | 0.007 | 0.05 |
| | | | | | color semi | | | | | | (RRT 0.767) | |
| | | | | | solid | | | | | | | |
| 12 | L | niclosamide + | Initial | | pale yellow | ND | ND | 0.028 | 0.010 | 0.006 | 0.010 | 0.06 |
| | | polyethylene | | | color liquid | | | | | | (RRT 0.139) | |
| | | glycol 400 | 40° C./75% RH | 4 Weeks | pale yellow | ND | ND | 0.027 | 0.011 | 0.009 | 0.007 | 0.05 |
| | | | | | color semi | | | | | | (RRT 0.766) | |
| | | | | | solid | | | | | | | |
| 13 | M | niclosamide + | Initial | | Off-White | ND | ND | 0.026 | 0.013 | 0.010 | 0.031 | 0.08 |
| | | glycerin | | | color semi | | | | | | (RRT 0.139) | |
| | | | | | solid | | | | | | | |
| | | | 40° C./75% RH | 4 Weeks | Off-White | ND | ND | 0.026 | 0.012 | 0.010 | 0.008 | 0.06 |
| | | | | | color semi | | | | | | (RRT 0.768) | |
| | | | | | solid | | | | | | | |

pH Adjusting Agents/Neutralizing Agents

| 14 | N | niclosamide + citric | Initial | | pale yellow | ND | ND | 0.029 | 0.011 | 0.011 | 0.013 | 0.10 |
|---|---|---|---|---|---|---|---|---|---|---|---|---|
| | | acid | | | color solid | | | | | | (RRT 0.139) | |
| | | | 40° C./75% RH | 4 Weeks | pale yellow | ND | ND | 0.027 | 0.012 | 0.009 | 0.007 | 0.06 |
| | | | | | color solid | | | | | | (RRT 0.765) | |
| 15 | O | niclosamide + lactic | Initial | | pale yellow | ND | ND | 0.026 | 0.010 | 0.010 | 0.010 | 0.06 |
| | | acid | | | color solid | | | | | | (RRT 0.139) | |
| | | | 40° C./75% RH | 4 Weeks | pale yellow | ND | ND | 0.027 | 0.012 | 0.010 | 0.007 | 0.06 |
| | | | | | color solid | | | | | | (RRT 0.766) | |
| 16 | P | niclosamide + sorbic | Initial | | pale yellow | ND | ND | 0.030 | 0.011 | ND | 0.014 | 0.06 |
| | | acid | | | color solid | | | | | | (RRT 0.768) | |
| | | | 40° C./75% RH | 4 Weeks | pale yellow | ND | ND | 0.027 | 0.013 | 0.009 | 0.006 | 0.06 |
| | | | | | color solid | | | | | | (RRT 0.766) | |
| 17 | Q | niclosamide + | Initial | | Pale pink | ND | 0.018 | 0.013 | 0.012 | 0.008 | 0.027 | 0.09 |
| | | sodium hydroxide | | | color solid | | | | | | (RRT 0.622) | |
| | | | 40° C./75% RH | 4 Weeks | Pale pink | 0.294 | 0.243 | 0.013 | 0.012 | 0.009 | 0.024 | 0.62 |
| | | | | | color solid | | | | | | (RRT 0.624) | |
| 18 | R | niclosamide + | Initial | | Orange red | ND | 0.004 | 0.006 | 0.012 | 0.009 | 0.026 | 0.07 |
| | | ethylene diamine | | | colored | | | | | | (RRT 0.621) | |
| | | | | | sticky solid | | | | | | | |
| | | | | | gel | | | | | | | |
| | | | 40° C./75% RH | 4 Week | Orange red | 0.003 | ND | 0.028 | 0.013 | 0.008 | 0.011 | 0.08 |
| | | | | | colored | | | | | | (RRT 0.615) | |
| | | | | | sticky solid | | | | | | | |
| | | | | | gel | | | | | | | |
| 19 | S | niclosamide + | Initial | | Pale yellow | ND | ND | 0.027 | 0.013 | 0.007 | 0.009 | 0.06 |
| | | potassium bitartrate | | | color powder | | | | | | (RRT 0.768) | |
| | | | 40° C./75% RH | 4 Week | Pale yellow | 0.002 | ND | 0.027 | 0.013 | 0.008 | 0.007 | 0.06 |
| | | | | | color powder | | | | | | (RRT 1.086) | |

Preservatives/Antioxidants

| 20 | T | niclosamide + benzyl | Initial | | pale yellow | ND | ND | 0.031 | 0.010 | 0.008 | 0.007 | 0.06 |
|---|---|---|---|---|---|---|---|---|---|---|---|---|
| | | alcohol | | | color semi | | | | | | (RRT 0.764) | |
| | | | | | liquid | | | | | | | |
| | | | 40° C./75% RH | 4 Week | pale yellow | ND | ND | 0.026 | 0.011 | 0.008 | 0.007 | 0.05 |
| | | | | | color semi | | | | | | (RRT 0.761) | |
| | | | | | solid | | | | | | | |
| 21 | U | niclosamide + benzoic | Initial | | Paly-yellow | ND | ND | 0.030 | 0.011 | ND | 0.012 | 0.05 |
| | | acid | | | to off white | | | | | | (RRT 0.163) | |
| | | | | | color solid | | | | | | | |
| | | | 40° C./75% RH | 4 Week | Paly-yellow | ND | ND | 0.026 | 0.001 | 0.008 | 0.007 | 0.05 |
| | | | | | color solid | | | | | | (RRT 0.767) | |
| | | | 40° C./75% RH | 4 Week | Pale yellow | 0.002 | ND | 0.028 | 0.014 | 0.008 | 0.007 | 0.06 |
| | | | | | color powder | | | | | | (RRT 1.086) | |

The impurities detected were as follows: Impurity-1 (5-chloro salicylic acid); Impurity-2 (2-chloro-4-nitro aniline); Impurity-3 (N-(2-chloro-4-nitrophenyl)-2-hydroxybenzamide); Impurity-4 (3,5-dichloro-N-(2-chloro-4-nitrophenyl)-2-hydroxybenzamide); and Impurity-5 (3-chloro-N-(2-chloro-4-nitrophenyl)-2-hydroxybenzamide).

Observations: No significant change in physical description was observed with any of the binary mixtures except with sodium CMC where color was changed from off-white to light pink. However, there was no impact on related substances observed. There was no significant change in related substances with any of the binary mixture even after exposure for 4 weeks except for combination of API with sodium hydroxide. In case of binary mixture containing sodium hydroxide the Total impurities increased significantly from 0.09% (Initial) to 0.62% (40° C./75% RH—4 weeks). Impurity-1 & Impurity-2 were increased upon exposure due to hydrolysis which is catalyzed by heat and alkali. Drug to NaOH ratio proposed to be used in the formulation for pH adjustment was very low. Impact of actual concentration in the formulation was monitored using short term stability study and based on the outcome usage of NaOH in the formulation was concluded. Based on the results described herein, all the gelling agents were considered for the formulation trials. Preservatives: benzoic acid and benzyl alcohol, Neutralizing agents: lactic acid, citric acid, sodium hydroxide and Solubilizers: PEG 400, were considered for further trials.

Preliminary Formulation Development Trials:

Described herein are initial formulation trials based on the preformulation data described herein, with the same formula with 3% and with increased drug concentration to 5%, as depicted in Table 9. The same formula was used for further trials with differing gelling agents and increased PEG concentration which are described herein.

TABLE 9
Qualitative and quantitative composition of formulation with
3% niclosamide and with 5% niclosamide [Batch Size: 150 g]

| Batch No.: | | 3% w/w niclosamide | | 5% w/w niclosamide | |
|---|---|---|---|---|---|
| Sr. | | Concentration | Quantity/Batch | Concentration | Quantity/Batch |
| No. | Ingredients | (% w/w) | (g) | (% w/w) | (g) |
| 1 | niclosamide | 3.00 | 4.50 | 5.00 | 7.50 |
| 2 | polyethylene glycol-400 | 35.00 | 52.50 | 35.00 | 52.50 |
| 3 | hydroxyethyl cellulose | 3.00 | 4.50 | 3.00 | 4.50 |
| | (Natrosol 250 HHX pharm) | | | | |
| 4 | potassium sodium tartrate | 0.35 | 0.525 | 0.35 | 0.525 |
| 5 | citric acid | 2.00 | 3.00 | 2.00 | 3.00 |
| 6 | lactic acid | 3.50 | 5.25 | 3.50 | 5.25 |
| 7 | benzoic acid | 0.20 | 0.30 | 0.20 | 0.30 |
| 8 | sodium chloride | 0.70 | 1.05 | 0.70 | 1.05 |
| 9 | sodium hydroxide | q.s. | q.s. | q.s. | q.s. |
| | (1N NAOH) | | | | |
| 10 | water | 50.55 | 75.83 | 48.55 | 72.90 |

Observations: Highly viscous, sticky, non-pourable, light yellow color opaque gel was formed.

Formulation trial with increased concentration of PEG-400 from 35% to 65% w/w: niclosamide was shown to have good solubility in PEG-400 (up to 7% w/w—from saturation solubility studies). Thus to solubilize 5% w/w of drug, concentration of PEG-400 was increased to 65% w/w in the formulation. 65% w/w of PEG-400 can be used for Topical formulations.

TABLE 10
Qualitative and quantitative composition of formulation with 5%
w/w of niclosamide and 65% w/w of PEG-400 [Batch Size: 150 g]

| Batch No.: | | 5% w/w niclosamide | |
|---|---|---|---|
| Sr. | | Concentration | Quantity/Batch |
| No. | Ingredients | (% w/w) | (g) |
| 1 | niclosamide | 5.00 | 7.50 |
| 2 | polyethylene glycol-400 | 65.00 | 97.50 |
| 3 | hydroxyethyl cellulose | 3.00 | 4.50 |
| | (Natrosol 250 HHX Pharm) | | |
| 4 | potassium bitartrate | 0.35 | 0.525 |
| 5 | citric acid | 2.00 | 3.00 |
| 6 | lactic acid | 3.50 | 5.25 |
| 7 | benzoic acid | 0.20 | 0.30 |
| 8 | sodium chloride | 0.70 | 1.05 |
| 9 | sodium hydroxide | q.s. | q.s. |
| | (1N NaOH) | | |
| 10 | water | 18.55 | 27.90 |

Observations: Highly viscous, non-pourable light yellow color opaque gel was formed.

Formulation Trial with Different Gelling Agents.

Formulation trials were done with various gelling agents with same formula and manufacturing process as used for testing the 5% w/w niclosamide formulation with 65% PEG-400, as described herein.

TABLE 11
Qualitative and quantitative composition of formulations with 5% w/w niclosamide, 65% w/w PEG-400 with different gelling
agents (Batch Size: 100 g)

| | | 5% w/w | | 5% w/w | | 5% w/w | | 5% w/w | |
|---|---|---|---|---|---|---|---|---|---|
| | | niclosamide with | | niclosamide with | | niclosamide with | | niclosamide with | |
| | | sodium CMC | | carbopol 980 | | pemulen TR-1 | | polycarbophil | |
| Formulation Types | | | Quantity/ | | Quantity/ | | Quantity/ | | Quantity/ |
| Sr. | | Concentration | Batch | Concentration | Batch | Concentration | Batch | Concentration | Batch |
| No. | Ingredients | (% w/w) | (g) | (% w/w) | (g) | (% w/w) | (g) | (% w/w) | (g) |
| 1 | niclosamide | 5.00 | 5.00 | 5.00 | 5.00 | 5.00 | 5.00 | 5.00 | 5.00 |
| 2 | polyethylene | 65.00 | 65.00 | 65.00 | 65.00 | 65.00 | 65.00 | 65.00 | 65.00 |
| | glycol-400 | | | | | | | | |
| 3 | sodium carboxy | 3.00 | 3.00 | — | — | — | — | — | — |
| | methyl cellulose | | | | | | | | |
| | (Blanose CMC | | | | | | | | |
| | 7HF PH) | | | | | | | | |
| 4 | carbopol 980 | — | — | 0.5 | 0.5 | — | — | — | — |
| 5 | pemulen TR-1 | — | — | — | — | 0.5 | 0.5 | — | — |
| 6 | polycarbophil | — | — | — | — | — | — | 0.5 | 0.5 |
| | (Noveon AA-1) | | | | | | | | |
| 4 | potassium | 0.35 | 0.35 | 0.35 | 0.35 | 0.35 | 0.35 | 0.35 | 0.35 |
| | bitartrate | | | | | | | | |
| 5 | citric acid | 2.00 | 2.00 | 2.00 | 2.00 | 2.00 | 2.00 | 2.00 | 2.00 |
| 6 | lactic acid | 3.50 | 3.50 | 3.50 | 3.50 | 3.50 | 3.50 | 3.50 | 3.50 |
| 7 | benzoic acid | 0.20 | 0.20 | 0.20 | 0.20 | 0.20 | 0.20 | 0.20 | 0.20 |
| 8 | sodium chloride | 0.70 | 0.70 | 0.70 | 0.70 | 0.70 | 0.70 | 0.70 | 0.70 |
| 9 | sodium | q.s. | q.s. | q.s. | q.s. | q.s. | q.s. | q.s. | q.s. |
| | hydroxide (1N | | | | | | | | |
| | NaOH) | | | | | | | | |
| 10 | water | 19.80 | 19.80 | 21.10 | 21.10 | 21.10 | 21.10 | 21.10 | 21.10 |

Observations: Viscous, slightly pourable light yellow color opaque gel was formed.

Physico-chemical evaluation of preliminary formulation trials of niclosamide gels: Preliminary formulation trials with different gelling agents were evaluated for description, assay & related substances as shown in Table 12.

TABLE 12
Physical and chemical evaluation of preliminary formulation trials of
niclosamide gel

| | YCT | YCT | Increased | | | | |
|---|---|---|---|---|---|---|---|
| | recipe with | recipe with | PEG-400 | sodium | carbopol | pemulen | |
| Tests | 3% API | 5% API | Concentration | CMC | 980 | TR-1 | polycarbophil |
| Description | Highly viscous, non-pourable | | | Viscous, slightly pourable light yellow color | | | |
| | light yellow color gel formed | | | gel formed | | | |
| Assay | Not Done | Done | Not Done | 101.2 | 96.2 | 95.6 | 95.2 |
| (% w/w) | | | | | | | |
| pH | 3.90 | 3.80 | 4.65 | 4.31 | 3.84 | 4.06 | 3.77 |

Related Substances (% w/w)

| | | | | | | | |
|---|---|---|---|---|---|---|---|
| Impurity-1 | ND | ND | ND | ND | ND | ND | 0.022 |
| Impurity-2 | ND | ND | ND | ND | ND | ND | ND |
| Impurity-3 | 0.028 | 0.035 | 0.028 | 0.026 | 0.027 | 0.027 | 0.027 |
| Impurity-4 | 0.012 | 0.010 | 0.013 | 0.011 | 0.013 | 0.014 | 0.012 |
| Impurity-5 | 0.010 | ND | 0.009 | 0.008 | 0.009 | 0.010 | 0.008 |
| Single | 0.008 | 0.013 | 0.007 | 0.007 | 0.006 | 0.006 | 0.006 |
| Maximum | (RRT-0.764) | (RRT-0.766) | (RRT-0.139) | (RRT-0.766) | (RRT-0.766) | (RRT-0.767) | (RRT-0.766) |
| Unknown | | | | | | | |
| Total | 0.06 | 0.06 | 0.06 | 0.05 | 0.06 | 0.06 | 0.08 |
| Impurities | | | | | | | |

Observations: Based on above data, it was concluded that there is no significant impact of various gelling agents on related substances of drug product.

Reproducible Formulation Trials with Various Gelling Agents:

Based on the physical observation of the preliminary formulation development trials described herein, few gelling agents were selected, Preservative benzoic acid was replaced with benzyl alcohol due to solubility issues of benzoic acid and reproducible formulation trials with different gelling agents were manufactured, packed in multilaminated tubes, submitted for initial evaluation and loaded for stability at conditions 40° C./75% RH (2W & 4W), 25° C./60% RH (2W & 4W), 60° C. (2W). Formulation composition details, physical and chemical evaluation are reported in Table 13.

TABLE 13
Qualitative and quantitative composition of reproducible
formulation trials of niclosamide gel

| | Batch Size: 250 g | | | | |
|---|---|---|---|---|---|
| | Quantity/Batch (g) | | | | |
| | Batch No. | | | | |
| | | | | 5% w/w | |
| | 3% w/w | 5% w/w | 5% w/w | API & | |
| | API & | API & | API & | 0.5% w/w | 5% w/w API |
| | 3% w/w | 3% w/w | 0.5% w/w | pemulen | & 3% w/w |
| Ingredients | HEC | HEC | carbopol | TR-1 | polycarbophil |
| niclosamide | 7.50 | 12.50 | 12.50 | 12.50 | 12.50 |
| polyethylene glycol-400 | 162.50 | 162.50 | 162.50 | 162.50 | 162.50 |
| hydroxyethyl cellulose | 7.50 | 7.50 | — | — | — |
| (Natrosol 250 HHX Pharm) | | | | | |
| carbopol 980 | — | — | 1.250 | — | — |
| pemulen TR-1 | — | — | — | 1.250 | — |
| polycarbophil (Noveon AA-1) | — | — | — | — | 1.250 |
| potassium bitartrate | 0.875 | 0.875 | 0.875 | 0.875 | 0.875 |
| citric acid | 5.00 | 5.00 | 5.00 | 5.00 | 5.00 |
| lactic acid | 8.750 | 8.750 | 8.750 | 8.750 | 8.750 |
| benzyl alcohol | 0.50 | 0.50 | 0.50 | 0.50 | 0.50 |
| sodium chloride | 1.75 | 1.75 | 1.75 | 1.75 | 1.75 |
| sodium hydroxide (1N NaOH) | 0.250 | 0.250 | 0.250 | 0.250 | 0.250 |
| water | 55.375 | 50.375 | 56.625 | 56.625 | 56.625 |
| Total | 250.00 | 250.00 | 250.00 | 250.00 | 250.00 |

TABLE 14
Physical and chemical evaluation of preliminary reproducible formulation
trials of niclosamide gel-initial formulations

| | | | | | Related Substances (%) | | | | | | |
|---|---|---|---|---|---|---|---|---|---|---|---|
| | | | | | | | | | | Maximum | |
| Tests | | | Viscosity | Assay | | | | | | Unknown | Total |
| Formulations | Description* | pH | (cPs) | (% w/w) | Imp-1 | Imp-2 | Imp-3 | Imp-4 | Imp-5 | Impurity | Impurities |
| 3% w/w API | Complies | 3.70 | — | 97.5 | ND | ND | 0.029 | 0.009 | 0.007 | 0.012 | 0.071 |
| & 3% w/w | | | | | | | | | | (RRT | |
| HEC | | | | | | | | | | 0.257) | |
| NCM-5-F- | Complies | 3.65 | 700 | 97.5 | 0.003 | ND | 0.030 | 0.014 | 0.009 | ND | 0.056 |
| 009 (5% w/w | | | | | | | | | | | |
| API & | | | | | | | | | | | |
| 3% w/w | | | | | | | | | | | |
| HEC) | | | | | | | | | | | |
| NCM-5-F- | Complies | 3.68 | 11000 | 104.1 | ND | ND | 0.027 | 0.009 | ND | 0.004 | 0.040 |
| 010 (5% w/w | | | | | | | | | | (RRT | |
| API & | | | | | | | | | | 1.066) | |
| 0.5% w/w | | | | | | | | | | | |
| carbopol) | | | | | | | | | | | |
| NCM-5-F- | Complies | 3.69 | 58000 | 102.4 | 0.002 | ND | 0.030 | 0.015 | 0.010 | 0.006 | 0.068 |
| 011 (5% w/w | | | | | | | | | | (RRT | |
| API & | | | | | | | | | | 0.761) | |
| 0.5% w/w | | | | | | | | | | | |
| pemulen TR- | | | | | | | | | | | |
| 1) | | | | | | | | | | | |
| NCM-5-F- | Complies | 3.71 | 18000 | 97.1 | 0.030 | ND | ND | 0.015 | 0.009 | 0.006 | 0.060 |
| 012 (5% w/w | | | | | | | | | | (RRT | |
| API & | | | | | | | | | | 0.764) | |
| 3% w/w | | | | | | | | | | | |
| polycarbophil) | | | | | | | | | | | |

*Pale yellow colored homogeneous opaque gel; ND—Not Detected

Observations: Viscosity data was found to be in increasing order as: HEC<carbopol <polycarbophil<pemulen base gels. Gel with HEC (700 cPs) as gelling agent had lowest viscosity and with pemulen TR-1 (58000 cPs) had highest viscosity. Based on above data, it was concluded that there is no significant impact of various gelling agents on pH and related substances of drug product.

Stability studies summary: Reproducible batches of niclosamide gel formulations described herein, were prepared and charged for stability at the accelerated (40±2° C. and 75±5% RH), long term (25±2° C. and 60±5% RH), and 60° C. The summary of data for the samples that were withdrawn is presented in Table 15. Product was observed to show satisfactory stability for all the batches studied in the multilaminated packs at stress studies and accelerated condition.

TABLE 15
Summary of stability study results of niclosamide gel formulations.

| | | | | Related Substances (%) | | | | | | |
|---|---|---|---|---|---|---|---|---|---|---|
| Tests | | | | | | | | | Max. | |
| | Testing | | | | | | | | Unk. | Total |
| Condition | Period | Description* | pH | Imp-1 | Imp-2 | Imp-3 | Imp-4 | Imp-5 | Impurity | Impurities |

Formulation: 3% API & 3% HEC

| Initial | | Complies | 3.70 | ND | ND | 0.029 | 0.009 | 0.007 | 0.012 | 0.071 |
|---|---|---|---|---|---|---|---|---|---|---|
| | | | | | | | | | (RRT | |
| | | | | | | | | | 0.257) | |
| 60° C. | 1 Week | Complies | — | 0.004 | 0.003 | 0.027 | 0.017 | ND | 0.006 | 0.073 |
| | | | | | | | | | (RRT | |
| | | | | | | | | | 1.054) | |
| | 2 Week | Complies | 3.68 | 0.008 | 0.004 | 0.029 | 0.013 | 0.006 | 0.009 | 0.092 |
| | | | | | | | | | (RRT | |
| | | | | | | | | | 0.575) | |
| 40° C./75% RH | 2 Week | Complies | — | 0.002 | ND | 0.028 | 0.013 | 0.008 | 0.008 | 0.063 |
| | | | | | | | | | (RRT | |
| | | | | | | | | | 1.087) | |
| | 4 Week | Complies | 3.54 | 0.004 | 0.002 | 0.028 | 0.009 | 0.007 | 0.004 | 0.054 |
| | | | | | | | | | (RRT | |
| | | | | | | | | | 0.773) | |

Formulation: 5% API & 3% HEC

| Initial | | Complies | 3.65 | 0.003 | ND | 0.030 | 0.014 | 0.009 | ND | 0.056 |
|---|---|---|---|---|---|---|---|---|---|---|
| 60° C. | 1 Week | Complies | — | 0.004 | 0.004 | 0.030 | 0.014 | ND | 0.003 | 0.055 |
| | | | | | | | | | (RRT | |
| | | | | | | | | | 0.284) | |
| | 2 Week | Complies | 3.68 | 0.009 | 0.004 | 0.030 | 0.014 | 0.008 | 0.007 | 0.093 |
| | | | | | | | | | (RRT | |
| | | | | | | | | | 0.768) | |
| 40° C./75% RH | 2 Week | Complies | — | 0.004 | ND | 0.028 | 0.013 | 0.008 | 0.005 | 0.062 |
| | | | | | | | | | (RRT | |
| | | | | | | | | | 1.085) | |
| | 4 Week | Complies | 3.61 | 0.006 | 0.002 | 0.027 | 0.010 | 0.008 | 0.004 | 0.057 |
| | | | | | | | | | (RRT | |
| | | | | | | | | | 0.772) | |

Formulation: 5% API & 0.5% carbopol-980

| Initial | | Complies | 3.68 | ND | ND | 0.027 | 0.009 | ND | 0.004 | 0.040 |
|---|---|---|---|---|---|---|---|---|---|---|
| | | | | | | | | | (RRT | |
| | | | | | | | | | 1.066) | |
| 60° C. | 1 Week | Complies | — | 0.004 | ND | 0.029 | 0.011 | ND | 0.019 | 0.083 |
| | | | | | | | | | (RRT | |
| | | | | | | | | | 1.055) | |
| | 2 Week | Complies | 3.90 | 0.010 | 0.003 | 0.029 | 0.020 | 0.008 | 0.019 | 0.131 |
| | | | | | | | | | (RRT | |
| | | | | | | | | | 0.575) | |
| 40° C./75% RH | 2 Week | Complies | — | 0.003 | ND | 0.028 | 0.012 | 0.008 | 0.005 | 0.059 |
| | | | | | | | | | (RRT | |
| | | | | | | | | | 1.087) | |
| | 4 Week | Complies | 3.27 | 0.004 | 0.001 | 0.028 | 0.010 | 0.012 | 0.005 | 0.062 |
| | | | | | | | | | (RRT | |
| | | | | | | | | | 0.772) | |

Formulation: 5% API & 0.5% pemulen TR-1

| Initial | | Complies | 3.69 | 0.002 | ND | 0.030 | 0.015 | 0.010 | 0.006 | 0.068 |
|---|---|---|---|---|---|---|---|---|---|---|
| | | | | | | | | | (RRT | |
| | | | | | | | | | 0.761) | |
| 60° C. | 1 Week | Complies | — | 0.003 | 0.005 | 0.028 | 0.011 | ND | 0.004 | 0.054 |
| | | | | | | | | | (RRT | |
| | | | | | | | | | 1.067) | |
| | 2 Week | Complies | 3.50 | 0.006 | 0.004 | 0.028 | 0.016 | 0.007 | 0.007 | 0.077 |
| | | | | | | | | | (RRT | |
| | | | | | | | | | 0.768) | |
| 40° C./75% RH | 2 Week | Complies | — | 0.003 | ND | 0.028 | 0.012 | 0.008 | 0.006 | 0.061 |
| | | | | | | | | | (RRT | |
| | | | | | | | | | 1.087) | |
| | 4 Week | Complies | 3.54 | 0.003 | 0.001 | 0.027 | 0.009 | 0.008 | 0.004 | 0.059 |
| | | | | | | | | | (RRT | |
| | | | | | | | | | 0.770) | |

Formulation: 5% API & 0.5% polycarbophil

| Initial | | Complies | 3.71 | 0.030 | ND | ND | 0.015 | 0.009 | 0.006 | 0.060 |
|---|---|---|---|---|---|---|---|---|---|---|
| | | | | | | | | | (RRT | |
| | | | | | | | | | 0.764) | |
| 60° C. | 1 Week | Complies | — | 0.003 | 0.005 | 0.004 | 0.015 | ND | 0.028 | 0.058 |
| | | | | | | | | | (RRT | |
| | | | | | | | | | 0.621) | |
| | 2 Week | Complies | 4.08 | 0.008 | 0.003 | 0.028 | 0.019 | 0.008 | 0.016 | 0.110 |
| | | | | | | | | | (RRT | |
| | | | | | | | | | 0.576) | |
| 40° C./75% RH | 2 Week | Complies | — | 0.003 | ND | 0.028 | 0.012 | 0.008 | 0.006 | 0.060 |
| | | | | | | | | | (RRT | |
| | | | | | | | | | 1.087) | |
| | 4 Week | Complies | 3.43 | 0.003 | 0.002 | 0.029 | 0.010 | 0.008 | 0.004 | 0.058 |
| | | | | | | | | | (RRT | |
| | | | | | | | | | 0.772) | |

*Pale yellow colored homogeneous opaque gel, ND—Not Detected, RRT—Relative Retention Time*

Observations: Related substances data was found to be almost similar at 60° C.—1W & 2W and 40° C./75% RH—2W & 4W when compared with initial data except for formulation batches with carbopol-980 and polycarbophil. In which related substances were increased at 60° C.—2W when compared with initial. Moreover, there was no significant impact on description and pH upon stability when compared with initial data.

Proposed Formula Composition and Process:

Based on the results obtained and inferences drawn from prototype formulation development, a qualitative and quantitative composition and manufacturing process proposed for further optimization and scale-up, as shown below.

TABLE 16
Qualitative and quantitative composition
for niclosamide gel 5% w/w

| | | Quantity |
|---|---|---|
| Name of | Concentration | per Gram |
| Ingredients | (% w/w) | (g) |
| niclosamide | 5.00 | 0.050 |
| polyethylene | 65.00 | 0.650 |
| glycol-400 | | |
| gelling agent* | 0.50 | 0.005 |
| potassium bitartrate | 0.35 | 0.0035 |
| citric acid | 2.00 | 0.020 |
| lactic acid | 3.50 | 0.035 |
| benzyl alcohol | 0.20 | 0.002 |
| sodium chloride | 0.70 | 0.007 |
| sodium hydroxide | 0.10 | 0.001 |
| purified water | 22.65 | 0.2265 |
| Total | 100 | 1.00 |

*Gelling agents would be selected from carbopol (0.5% w/w), pemulen TR-1 (0.5% w/w), polycarbophil (0.5% w/w), HEC (3% w/w)

Provided herein is a manufacturing/preparation process for the niclosamide formulations disclosed herein, as follows: 1. In the first vessel, niclosamide can be mixed in PEG-400 for 30 minutes at 500-600 RPM using overhead stirrer. It can be further solubilised with simultaneous heating and stirring for 30 min. benzoic acid and benzyl alcohol can be added to the solution and solubilised by stirring; 2. In a separate vessel 20 g water, sodium chloride, citric acid, lactic acid, potassium tartrate and carbopol 980 can be combined and mixed for 15 minutes at 100-200 RPM using overhead stirrer; 3. Aqueous fraction of step 3 can be added to the PEG fraction of step 2 under stirring and mixing vigorously for 15 minutes at 900-1200 RPM using overhead stirrer; 4. 1N NaOH can be added to adjust the pH to ˜3.70. (˜2.5 mL and for pH found to be 3.80 of ˜3.70); 5. Remaining water (7.90 g) can be added to get the final volume; 6. Homogeneity can be achieved by mixing for 10 minutes at 500-600 RPM using overhead stirrer.

### Example 3

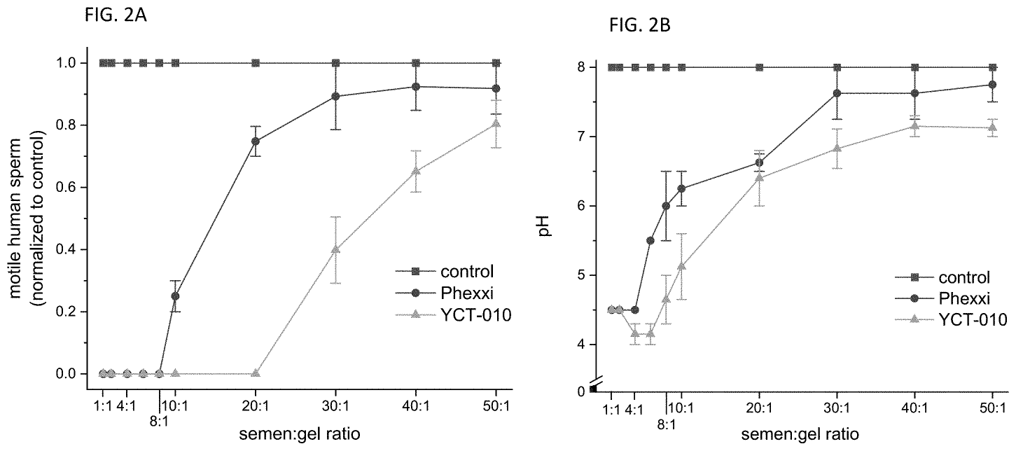

Described herein is a study comparing the efficacy of the niclosamide formulation disclosed herein with Phexxi, a commercially available contraceptive gel from Evofemin, in inhibiting sperm motility and in pH buffering capacity. Fresh human semen was mixed with niclosamide gel formulation of the disclosure, at different ratios, and human sperm motility and pH were measured. Results described herein, show that the the niclosamide gel formulation of the disclosure, maintained complete inhibition of sperm cells in human semen at semen to gel ratio of 20:1. In contrast, incubation of human semen with Phexxi resulted in motile sperm cells at a semen to semen to gel ratio of 10:1 (FIG. 2A). Further, as depicted in FIG. 2B, the pH buffering capacity (ability to maintain pH between 3-5), of the niclosamide gel formulation of the present disclosure is significantly higher than that of Phexxi, at the same semen to gel ratio (4:1 to 10:1). Based on the results described herein, the niclosamide gel formulation of the present disclosure, is more effective in inhibiting sperm motility and pH buffering capacity, as compared to commercially available contraceptive formulations.

### Example 4

Described herein is a study to determine drug release over time from the niclosamide gel formulation, as disclosed herein. Two initial niclosamide gel formulations (Formulation A and Formulation B) described in Table 17 below, were covered with vaginal fluid simulant (VFS) and incubated at 37° C. At time points t=0, 5, 10, 30 and 60 minutes, VFS was aspirated and analyzed by LC-MS/MS to detect niclosamide.

TABLE 17
Drug and excipient concentrations of niclosamide gel
formulations A and B, used in drug release study.

| | % w/w | grams |
|---|---|---|
| Formulation A | | |
| NaCl | 0.795731136 | 0.085 |
| lactic acid | 9.361542782 | 1 |
| water | 65.53079948 | 7 |
| 1N NaOH | 18.72308556 | 2 |
| HEC | 2.527616551 | 0.27 |
| NA | 3.06122449 | 0.327 |
| Total | 100 | 10.682 |
| Formulation B | | |
| NaCl | 0.79573 | 0.085 |
| lactic acid | 9.36154 | 1 |
| water | 56.1693 | 6 |
| 1N NaOH | 18.7231 | 2 |
| HEC | 2.52762 | 0.27 |
| PEG-400 | 9.36154 | 1 |
| NA | 3.06122 | 0.327 |
| Total | 100 | 10.682 |

TABLE 18
Drug release from niclosamide gel formulations A and B.

| | | |
|---|---|---|
| | niclosamide | |
| time | released (mM) | |
| (minutes) | Formulation A | Formulation B |

| | | | |
|---|---|---|---|
| 0 | min | 0.039 | <LOD |
| 5 | min | 0.034 | <LOD |
| 10 | min | 0.057 | <LOD |
| 30 | min | <LOD | <LOD |
| 60 | min | 2.2 | 7.6 |

<LOD: Below the limit of detection

Conclusion: Data from the study shows niclosamide release from the gel formulations of the disclosure.

### Example 5

Described herein is a study to assess and evaluate anti-fertility effects of niclosamide in rabbits. For this study, New Zealand White rabbits, comprising of 15-30 females (does) and 15-30 male (bucks), that are healthy, weighing 1.5-3.0 kg and of age 32-40 weeks will be used. All the animals will be randomized based on body weight, into three study groups: native control, control treatment and niclosamide treatment. Male rabbits of proven fertility will be used for mating both the control and test treated female rabbits. To ensure ovulation, 100 i.u. of human chorionic gonadotrophin (Fertigyn Hp-5000 5000 IU Injection; Sun Pharma Laboratories Ltd.) will be administered through the marginal ear vein of each participating doe 2 hour prior to mating. 1 mL of niclosamide-based gel will be administered vaginally to does. Does will be administered with respective treatment as shown in Table 19, through intra vaginal route 1 hour prior to mating. To ensure even distribution of the gel in the vaginal vault, does will be held in a supine position for about 10 minutes. Approximately, 20 minutes after gel application, a buck of proven fertility will be introduced to each doe for mating. Does and bucks will be separated after mating and does will be euthanized after 30 days. Body weights and clinical signs of mating will be monitored regularly. The vaginal lavage/swab of the mated female rabbit will be examined approximately 4 hours after mating under a microscope for the presence of sperm, using standard procedures. Blood will be collected from central ear artery of the bucks after second phase of mating at about 2 hrs (As soon as mating will be completed) and 4 hrs. End point: After 10 days of mating, female rabbits will be euthanized using thiopental sodium at dose of 100 mg/kg body weight and number of implants will be counted on both uterine horns following incising at abdominal region, to determine efficacy of the administered dose of niclosamide gel formulation in in the treatment group as compared to the control groups.

TABLE 19
Study design of rabbit mating study

| | | | Compound | | |
|---|---|---|---|---|---|
| | | No. of | Concentration | Dose Volume | Administration |
| Group | Treatment | Animals | per Animal | per Animal | route |
| 1 | Naive Control | 5-10F/5-10M | — | — | Intra vaginal |
| 2 | Vehicle Control | 5-10F/5-10M | — | 1 | Intra vaginal |
| 3 | niclosamide | 5-10F/5-10M | 150 mM | 1 | Intra vaginal |

### Example 6

Described herein is the design of a blinded and randomized crossover, phase 1 clinical study to determine evaluate safety and contraceptive properties of a niclosamide gel formulations of the disclosure. A total of about 30 couples will be enrolled for this study.

Study description and intervention: The test product will be niclosamide gel at 150 mM. Phexxi™ will be used as reference therapy. Each woman of the enrolled couples, will have four cycles under study: the first for a baseline PCT and the following three for receiving pre-intercourse treatment (niclosamide 150 mM or Phexxi™) according to randomization. The first PCT will be a baseline test without the use of any product, in order to demonstrate that the subject experiences normal ovulatory events, produces receptive midcycle cervical mucus, and has a partner who is able to produce motile sperm capable of penetrating midcycle mucus. The treatment will then be provided according to the randomization order. niclosamide gel formulation (API and excipients, and their concentrations) to be used for study: Details of the niclosamide formulation used in this study are provided in Table 20.

TABLE 20
Components and concentrations of API and
excipients of the final formulation.
Components of the niclosamide vaginal gel

| Item No. | Ingredient | Concentration (% w/w) |
|---|---|---|
| 1 | niclosamide | 5.00 (153 mM) |
| 2 | polyethylene glycol-400 | 65.00 |
| 3 | carbopol 980 | 0.50 |
| 4 | potassium sodium tartrate | 0.35 |
| 5 | citric acid | 2.00 |
| 6 | lactic acid | 3.50 |
| 7 | benzyl alcohol | 0.20 |
| 8 | sodium chloride | 0.70 |
| 9 | sodium hydroxide (1N) | 0.10 |
| 10 | water | 22.65 |

Final pH = 3.65-3.70

With niclosamide dissolved in DMSO, a spermicidal effect at ≥10 mM was observed. With niclosamide in formulation, a spermicidal effect at a final concentration between 5 mM and 7.5 mM or higher, was observed. The effective range for niclosamide was determined to be from 5-150 mM. Ranges of: A spermicidal effect at ≥50% (v/v/) was observed when mixing polyethylene glycol-400 (PEG-400) with fresh human semen. The effective range for PEG-400 was determined to be from 50-65%.

Screening, baseline, and treatment evaluations: On the day of screening, a urine pregnancy test, physical examination, pelvic exam, and bacterial vaginosis (BV) assessment (by Amsel's criteria) will be performed. Samples for Pap test, Gram stain (Nugent criteria), endocervical gonorrhea culture, and endocervical chlamydia ELISA test will be collected, and diagnosis of any STI and appropriate treatment will be done, before considering the volunteer for participation. Each woman will receive an adequate supply (e.g., 15 or more) of non-spermicidal lubricated condoms for use until 72 h before her midcycle visit and will be asked to keep record of her menstrual days, coitus, and condom use. An appointment will be scheduled on the expected midcycle day of the next menstrual cycle, based on the woman's two previous menstrual cycles. Volunteers will be contacted at the estimated date of their next menstrual period to confirm the appointment.

Upon confirming the first day of the cycle, a visit for mucus check will be scheduled for day 12. At that visit, samples of cervical and vaginal secretions will be examined for midcycle characteristics and the presence of sperm. If no sperm are detected on this visit and if the mucus showed midcycle characteristics, the volunteer will be instructed to return home, to have sex without a condom the following morning, and to come to the clinic within 2 h to perform a PCT.

During the PCT visit, samples from the vaginal pool and cervical mucus will be collected within 2 h following coitus and placed on two separate slides to be directly evaluated under microscope, following the World Health Organization's (WHO's) PCT guidelines. Samples will be evaluated for midcycle characteristics, the presence of sperm, and the quantity and motility of the sperm in the cul-de-sac and endocervix, no later than 30 min after collection. Slides will be reviewed in two stages: first, the entire slide to detect the presence of any sperm (100×), and second, nine high-power fields (400×) evenly distributed.

In PCTs carried out during treatment cycles, the following will be recorded and confirmed: a) compliance with the protocol schedule, b) interval from study gel application to coitus and from coitus to sample collection, and c) symptoms reported by each member of the couple after use of the study gel and during coitus, including excessive lubrication, partner's complaints, and any other adverse effects (AEs).

End point: A 72-h postcoital visit will be scheduled to collect vaginal swab and cervicovaginal lavage for the evaluation of potential effects on the vaginal ecology and inflammation by laboratory markers (H2O2-producing lactobacillus, vaginal Gram stain by Nugent criteria, IL-6, leukocytes count and differential). After the last 72-h postcoital visit (Cycle 4), or earlier if applicable, the subject will be exited from the study.

### Example 7

Described herein is a study to to assess and evaluate vaginal irritation effect of Niclosamide. A total of 9 female New Zealand white rabbits were used for the study described herein. The animals were divided into three groups of three animals in each group: Group 1 (G1) animals treated instilled/treated vehicle control; Group 2 (G2) animals instilled/treated with 2.5 mM dose of niclosamide; and Group 3 (G3) animals instilled/treated with 100 mM dose of niclosamide, as shown in Table 21.

TABLE 21
Study design description

| | | | | Dose | No. of | Animal |
|---|---|---|---|---|---|---|
| Group | Treatment | | | volume | Animals | No. |
| No. | Group | Doses | | mL/animal | Female | Female |
| G1 | Vehicle Control | — | | 1.0 | 3 | 1-3 |
| G2 | Niclosamide | 2.5 | mM | 1.0 | 3 | 4-6 |
| G3 | Niclosamide | 100 | mM | 1.0 | 3 | 7-9 |

Formulations preparation used for the study were as follows. Vehicle Preparation: 0.85% sodium chloride, 0.1% sorbic acid, 10% lactic acid and 75% water was taken in mortar and pestle and triturated, pH was adjusted to approximately 4.5 using 10 N sodium hydroxide. 2.7% natrosol was added and triturated. Remaining volume was adjusted with water. Total quantity was triturated again and formulation pH was confirmed with pH paper. Niclosamide formulation preparation: 0.85% sodium chloride, 0.1% sorbic acid, 10% lactic acid and 75% water was taken in mortar and pestle and triturated. pH was adjusted to approximately 4.5 using 10 N sodium hydroxide. Required amount of Niclosamide and 2.7% Natrosol was added and triturated. Remaining volume was adjusted with water. Total quantity was triturated again and formulation pH was confirmed with pH paper.

Instillation of the treatment was done using prefilled FG-08 tube attached to 1 mL disposable syringe at a dose volume of 1.0 mL per animal continuously for 7 days (from day 1 to 7). Irritation potential was scored in all animals using draize scale daily before Niclosamide instillation and approximately 30 minutes after Niclosamide instillation daily from day 1 to 7 and 24 hours after last test item instillation (day 8). Animals were observed for clinical signs, including morbidity and mortality. All animals were weighed on the day of receipt, prior to randomization, on day 1 of dosing and on day 4, 7 and 8. Blood samples were collected (approximately 1.2 mL) at 0 (pre dose on day 7), and 1, 3, 6, 12 hours (day 7) and at 24 hours (day 8) after last vaginal instillation from all animals. On day 8 animals were euthanized and subjected to gross pathological examination and organs were collected (Cervical, central and caudal parts of the vagina) in 10% neutral buffer formalin solution for histopathological evaluation.

Results:

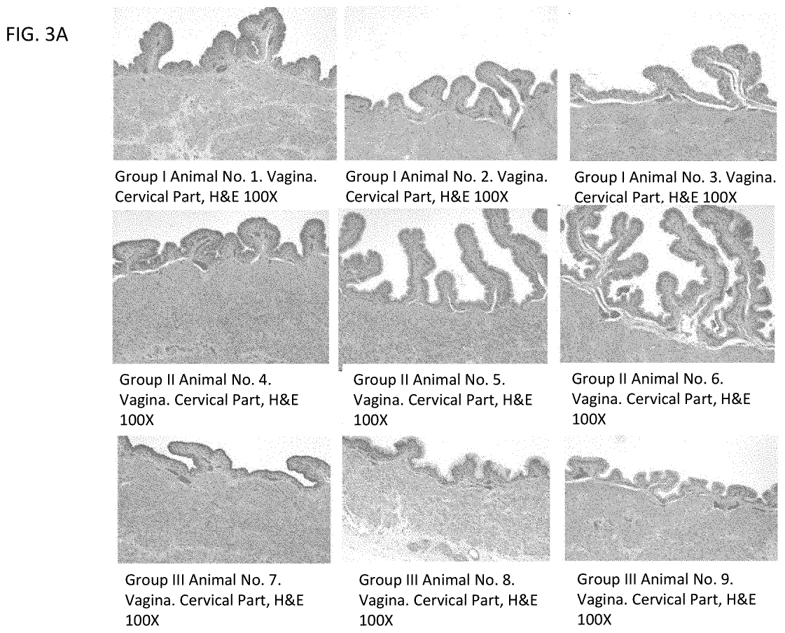

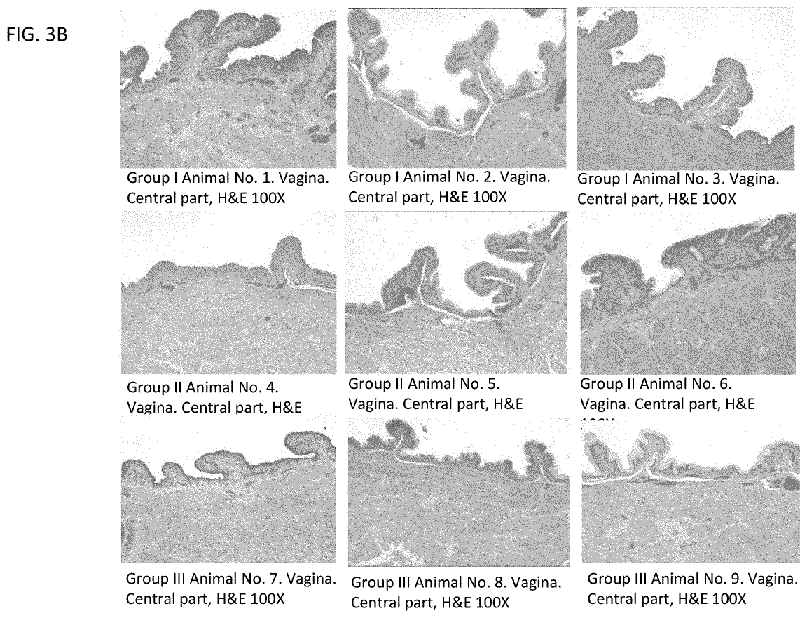

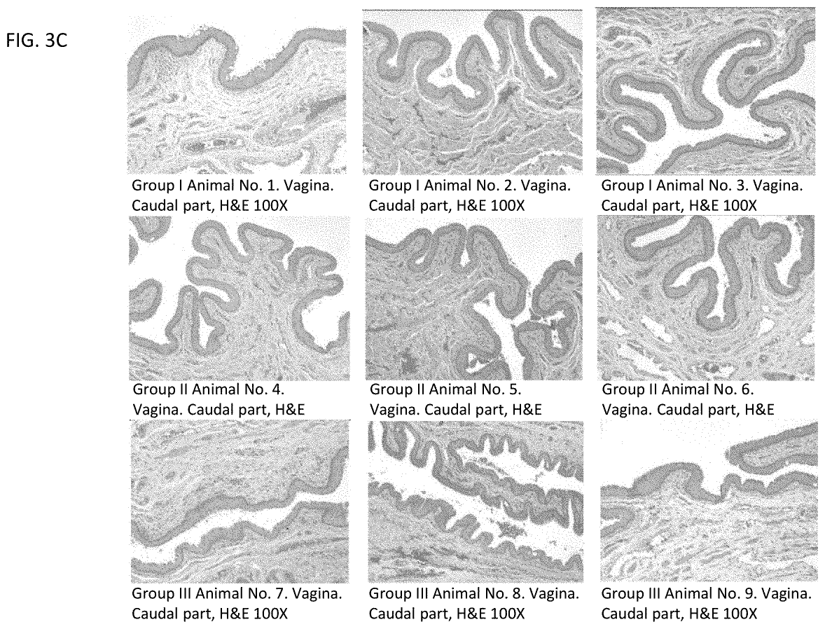

Clinical Signs and Mortality: No treatment related mortality and adverse clinical signs (including erythema and oedema) and were observed throughout the experiment across all other animals. All animals were found normal in detailed clinical examination. No irritation related to Niclosamide was observed in any of the animals. Body Weight: Body weight gain was observed in all animals throughout the study described herein. Gross Pathology: Vagina was reddened at central lower half to caudal area mildly in all animals belonging to all groups, showing that the observation cannot be attributed to test item. Histopathology: No treatment related Histopathological findings were observed, as shown in FIGS. 3A-3C. Mild congestions were observed in all the parts of vagina of both vehicle and treatment groups. These findings were typical of those that occur as a result of the repeated insertion of the catheter. Plasma Concentration: Plasma concentrations were observed at early time points between 1 to 6 h after dosing on day 7 in both treatment groups of the Niclosamide, as shown in Table 22.

TABLE 22
Summary of Plasma Concentrations on Day 7

| | | | Time | | | | | |
|---|---|---|---|---|---|---|---|---|
| | Compound | | (hours) | Concentrations (ng/mL) | | | | |
| Group | ID | Dose | points | Ani#1 | Ani#2 | Ani#3 | AVG | SEM |
| 2 | Niclosamide | 2.5 mM | 0 | 0.00 | 0.00 | 0.00 | 0.00 | 0.00 |
| | | (0.823 mg/animal) | 1 | 1.33 | 1.19 | 1.48 | 1.33 | 0.09 |
| | | | 3 | 0.00 | 1.65 | 0.74 | 0.80 | 0.48 |
| | | | 6 | 0.00 | 0.54 | 0.27 | 0.27 | 0.16 |
| | | | 12 | 0.00 | 0.00 | 0.00 | 0.00 | 0.00 |
| | | | 24 | 0.00 | 0.00 | 0.00 | 0.00 | 0.00 |
| 3 | | 100 mM | 0 | 0.00 | 0.00 | 0.00 | 0.00 | 0.00 |
| | | (32.91 mg/animal) | 1 | 3.38 | 1.41 | 12.46 | 5.75 | 3.40 |
| | | | 3 | 1.55 | 5.80 | 0.92 | 2.76 | 1.53 |
| | | | 6 | 1.33 | 0.00 | 0.70 | 0.68 | 0.38 |
| | | | 12 | 0.00 | 0.00 | 0.00 | 0.00 | 0.00 |
| | | | 24 | 0.00 | 0.00 | 0.00 | 0.00 | 0.00 |

Conclusion: Based on the above results, the test item Niclosamide has no irritation effect on vagina of rabbits under the experimental conditions tested.

### Equivalents

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically aIs nd individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Unless indicated otherwise, all percentages by are percentages by weight, parts are parts by weight, temperature is in degrees Celsius, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

1. A formulation comprising an amount of niclosamide and one or more of a humectant, a lubricant, a solvent, an osmolality modulator, a pH modulator, a viscosity modulator, a preservative, or combinations thereof.

2. The formulation of claim 1, wherein the formulation has a viscosity of between about 700 cPs and about 58,000 cPs, or between about 10,000 cPs and about 18,000 cPs, or about 11,000 cPs, wherein optionally the viscosity modulator is HEC, or carbopol, or polycarbophil, or pemulen.

3-7. (canceled)

8. The formulation of claim 1, wherein the humectant, the lubricant or the solvent comprises a synthetic polymer.

9. The formulation of claim 8, wherein the humectant, the lubricant or the solvent comprises at least two monomers of polyethylene glycol (PEG), optionally PEG-400, PEG-1000, or PEG-2000.

10. The formulation of claim 8, wherein the humectant, the lubricant or the solvent comprises PEG-400.

11. The formulation of claim 1, wherein the formulation comprises an amount of PEG-400 between 0.1% and 75%, between 35% and 75%, between 35% and 65%, about 65%, or 65% of the total weight of the formulation, inclusive of the endpoints.

12-15. (canceled)

16. The formulation of claim 1, wherein the formulation comprises a semisolid form, a gel, or a cream.

17. (canceled)

18. The formulation of claim 1, wherein the amount of niclosamide is between 0.1% and 10%, between 3% and 7%, between 3% and 6%, between 3% and 5%, about 5%, or 5% of total weight of the formulation, inclusive of the endpoints.

19-23. (canceled)

24. The formulation of 1, wherein the niclosamide has a concentration of between 4 mM and 500 mM, between 10 mM and 250 mM, about 150 mM, or 150 mM, inclusive of the endpoints.

25-27. (canceled)

28. The formulation of claim 1, wherein the solvent comprises water and wherein formulation comprises an amount of the water of between 10% and 90% of the total weight of the formulation, inclusive of the endpoints.

29. The formulation of claim 1, wherein the osmolality modulator comprises sodium chloride.

30. The formulation of claim 1, wherein the formulation comprises an amount of the osmolality modulator of less than 5% or less than 1% of the total weight of the formulation.

31. (canceled)

32. The formulation of claim 1, wherein the pH modulator comprises one or more of lactic acid, potassium sodium tartrate, citric acid monohydrate, potassium bitartrate and sodium hydroxide.

33. The formulation of claim 1, wherein the formulation comprises an amount of the pH modulator of less than 10%, less than 5%, less than 1%, or less than 0.5% of the total weight of the formulation, and wherein the pH modulator comprises one or more of potassium sodium tartrate, citric acid monohydrate, potassium bitartrate, and sodium hydroxide.

34-36. (canceled)

37. The formulation of claim 1, wherein the viscosity modulator is a viscosity enhancer, optionally the viscosity enhancer comprising one or more of hydroxyethyl cellulose, alginic acid, polycarbophil and carbopol, wherein the formulation comprises an amount of a viscosity enhancer of between 1% and 10%, between 1% and 5%, less than 5%, 3%, less than 1%, about 0.5%, or 0.5% of the total weight of the formulation, inclusive of the endpoints.

38-49. (canceled)

50. The formulation of claim 1, wherein the preservative comprises one or more of benzyl alcohol, chlorhexidine gluconate and benzoic acid.

51-52. (canceled)

53. The formulation of claim 1, wherein the formulation comprises an amount of the preservative of less than 1%, about 0.2%, 0.2%, or less than 0.2% of the total weight of the formulation.

54-56. (canceled)

57. The formulation of claim 1, wherein the formulation comprises, a) niclosamide at a concentration of 3% to 5% by weight of the formulation;
b) a humectant, a lubricant or a solvent, wherein the humectant, the lubricant or the solvent comprises an amount of PEG-400 of 35% to 65% by weight of the formulation and an amount of water of 10% to 60% by weight of the formulation;
c) an osmolality modulator, wherein the osmolality modulator comprises an amount of sodium chloride of 0.1% to 1% by weight of the formulation;
d) at least one pH modulator, wherein the at least one pH modulator comprises an amount of lactic acid of 1% to 5% by weight of the formulation, an amount of citric acid monohydrate of 1% to 5% by weight of the formulation, an amount of potassium sodium tartrate of 0.1% to 0.5% by weight of the formulation, and an amount of sodium hydroxide of 0.1% to 0.5% by weight of the formulation;
e) at least one viscosity modulator, wherein the at least one viscosity modulator comprises an amount of carbopol of 0.1% to 1% by weight of the formulation; and
f) at least one preservative, wherein the at least one preservative comprises an amount of benzyl alcohol of 0.1% to 0.5% by weight of the formulation.

58. The formulation of claim 1, wherein the formulation comprises,
a) niclosamide at a concentration of 100 mM;
b) a humectant, a lubricant or a solvent, wherein the humectant, the lubricant or the solvent comprises an amount of PEG-400 of between 0.1% and 65% by weight of the formulation and an amount of water of between 10% and 90% by weight of the formulation;
c) an osmolality modulator, wherein the osmolality modulator comprises an amount of sodium chloride of less than 1% by weight of the formulation;
d) at least one pH modulator, wherein the at least one pH modulator comprises an amount of lactic acid of less than 10% by weight of the formulation, an amount of citric acid monohydrate of less than 5% by weight of the formulation, an amount of potassium bitartrate of less than 0.5% by weight of the formulation, and an amount of sodium hydroxide of less than 1% by weight of the formulation;
e) at least one viscosity modulator, wherein the at least one viscosity modulator comprises an amount of hydroxyethyl cellulose of 3% by weight of the formulation, an amount of alginic acid of less than 5% by weight of the formulation, an amount of polycarbophil of less than 5% by weight of the formulation and an amount of carbopol of less than 5% by weight of the formulation; and
f) at least one preservative, wherein the at least one preservative comprises an amount of benzyl alcohol of less than 1% by weight of the formulation, an amount of chlorhexidine gluconate of less than 0.12% by weight of the formulation, and an amount of benzoic acid of less than 0.2% by weight of the formulation.

59. A method of contraception, comprising administering an effective amount of the formulation of claim 1, to the subject, wherein, upon contacting a sperm cell, the formulation inhibits or decreases one or more of sperm motility, sperm viability, and sperm metabolism, thereby decreasing the probability of conception.

60. A method of decreasing the probability of conception in a female subject in need thereof, comprising contacting the female subject with an effective amount of the formulation of claim 1, intravaginally prior to a sexual intercourse or coitus, wherein the probability of conceiving post the sexual intercourse or coitus of the female subject contacted with the formulation, is less than the probability of conceiving post the sexual intercourse or coitus of the female subject that has not been contacted with the formulation.

61. The formulation according to claim 1, for use in promoting contraception, comprising administering an effective amount of the formulation to the subject, wherein, upon contacting a sperm cell, the formulation inhibits or decreases one or more of sperm motility, sperm viability, and sperm metabolism, thereby decreasing the probability of conception.

62. A method of making a niclosamide formulation, wherein the method comprises:
a) combining an amount of niclosamide with a amount of PEG-400 in a first vessel to form a solution;
b) combining an amount of benzoic acid and/or benzyl alcohol with the solution of (a) to form a PEG fraction;
c) combining an amount of water, an amount of sodium chloride, an amount of citric acid, an amount of lactic acid, an amount of potassium tartrate and an amount of carbopol 980 in a separate vessel to form an aqueous fraction;
d) combining the aqueous fraction of (c) with the PEG fraction of (b);
e) adding an amount of sodium hydroxide to the mixture of (d) to adjust the pH;
f) optionally adding water to the mixture of (e); and
g) mixing the mixture of (e) or (f) till a homogenous mixture is obtained.

63. The method of claim 62, wherein step (a) comprises mixing by stirring for about 30 minutes at a speed of between 500-600 rotations per minute (RPM) and optionally step (a) further comprises stirring the mixture with simultaneously heating and stirring.

64. (canceled)

65. The method of claim 63, wherein the heating of the mixture is done to a temperature of about 65° C., optionally step (c) comprises mixing by stirring for about 15 minutes at a speed of between 100-200 RPM, optionally step (d) comprises mixing by stirring for about 15 minutes at a speed of between 900-1200 RPM, and optionally step (g) comprises mixing by stirring for about 10 minutes at a speed of between 500-600 RPM.

66-67. (canceled)
